(19) United States
(12) Patent Application Publication
(10) Pub. No.: US 2025/0059506 A1
(43) Pub. Date: Feb. 20, 2025

(54) LABEL-FREE CELL ISOLATION

(71) Applicant: ZEON CORPORATION, Chiyoda-ku Tokyo (JP)

(72) Inventors: Anthony WARD, Richmond, VA (US); Roberto CAMPOS GONZALEZ, Richmond, VA (US); Alison SKELLEY, Richmond, VA (US); Yasna BEHMARDI, Richmond, VA (US); Khushroo GANDHI, Richmond, VA (US); Mabel SHEHADA, Richmond, VA (US)

(73) Assignee: ZEON CORPORATION, Chiyoda-ku Tokyo (JP)

(21) Appl. No.: 18/719,831

(22) PCT Filed: Dec. 15, 2022
(86) PCT No.: PCT/US2022/081675

§ 371 Date: Jun. 14, 2024

Related U.S. Application Data
(60) Provisional application No. 63/289,982, filed on Dec. 15, 2021.

(51) Int. Cl. C12N 5/078; C12N 5/00

(52) U.S. Cl. CPC C12N 5/0634; C12N 5/0087; C12N 2501/2307; C12N 2501/2315

(57) ABSTRACT

Described herein is a method of enriching lymphocytes from a biological sample, the method comprising: (a) removing large cells and small cells from the biological sample by a size based selection method to obtain lymphocytes, wherein the large cells comprise granulocytes or monocytes, or a combination thereof, and the small cells comprise platelets, red blood cells, or a combination thereof; (b) contacting the lymphocytes with an activating agent to obtain activated lymphocytes; and (c) removing inactivated lymphocytes from the activated lymphocytes using a sized based selection method to obtain activated enriched lymphocytes, thereby obtaining enriched activated lymphocytes.

### CROSS-REFERENCE

This application claims the benefit of U.S. Provisional App. No. 63/289,982 filed on Dec. 15, 2021, which is incorporated by reference herein in its entirety.

### SUMMARY

Current approaches to autologous and allogeneic based cellular therapy manufacture typically include recovery of the cell to be engineered, activating the cells to initiate cell division, and then using one or more approaches to insert sequence to enable bioengineering for therapeutic effect, then expanding the cells, or not, as needed to produce enough therapeutic agent. To achieve selection of the specific cell types, an immunoselection strategy is typically employed, either via positive selection (desired cell specific) or via negative selection (removal of undesired cells) via an affinity reagent such as an antibody targeting a desired cell. Such affinity reagents are typically conjugated to a particle. Often these particles allow selection via a magnetic or paramagnetic cell selection scheme. Such schemes are generally around 60-70% efficient from an unprocessed matrix. In a manufacturing process, the addition of paramagnetic, magnetic or other particles to a parenteral drug product manufacturing scheme are problematic to the process, as regulatory bodies require full documentation of the removal of those particles from the drug product. The cost contribution of such particles is a considerable driver of costs in the development and manufacture of a typical therapy, as they require full documentation of their removal in addition to the initial expense of the particles in the first selection step.

Described herein are methods of selecting and isolating lymphocytes for cell therapeutic applications that reduce or eliminate the need for costly and laborious particle or label based selection.

Described herein is a method of enriching lymphocytes from a biological sample, the method comprising: (a) separating large cells and small cells from the biological sample by a size based selection method to obtain lymphocytes, wherein the large cells comprise granulocytes or monocytes, or a combination thereof, and the small cells comprise platelets, red blood cells, or a combination thereof; (b) contacting the lymphocytes with a size increasing agent to obtain size increased lymphocytes; and optionally (c) separating non-size increased lymphocytes from the size increased lymphocytes to obtain size increased enriched lymphocytes, thereby obtaining enriched size increased lymphocytes.

In certain embodiments, the method further comprises separating platelet derived microvesicles, microparticles, or exosomes. In certain embodiments, the biological sample is a blood related sample. In certain embodiments, the biological sample is an apheresis product. In certain embodiments, the apheresis product is a leukapheresis product.

In certain embodiments, the large cells possess a diameter of 8.5 micrometers or greater. In certain embodiments, the large cells possess a diameter of 6.5 micrometers or greater. In certain embodiments, the large cells possess a diameter of 8 micrometers or greater, 7.5 micrometers or greater, 7 micrometers or greater, or 6.5 micrometers or greater. In certain embodiments, the large cells comprise monocytes, granulocytes, dendritic cells, or a combination thereof. In certain embodiments, the small cells possess a dimeter of 3.5 micrometers or less. In certain embodiments, the small cells possess a diameter of 4 micrometers or less, 4.5 micrometers or less, or 5 micrometers or less.

In certain embodiments, the small cells comprise platelets and red blood cells. In certain embodiments, the size increasing agent comprises an activating agent. In certain embodiments, the lymphocytes comprise T cells, B cells, NK cells, or a combination thereof. In certain embodiments, the lymphocytes comprise T cells. In certain embodiments, the size increasing agent is selected from the list consisting of a CD3 binding antibody, a CD28 binding antibody, a CD49d binding antibody, Concanavalin-A, and combinations thereof. In certain embodiments, the lymphocytes comprise B cells. In certain embodiments, the size increasing agent is selected from the list consisting of a IgM binding antibody, a IgD binding antibody, a CD154 binding antibody, a CpG oligonucleotide, LPS, single stranded RNA, imiquimod, and combinations thereof. In certain embodiments, the lymphocytes comprise NK cells. In certain embodiments, the size increasing agent is selected from the list consisting of a CD335(NKp46) binding antibody, a CD2 binding antibody, LPS, peptidoglycan, a mIR-150 microRNA, and combinations thereof.

In certain embodiments, the non-size increased lymphocytes possess a diameter of 6.5 micrometers or less. In certain embodiments, the non-size increased lymphocytes possess a diameter of 4.2 micrometers or less. In certain embodiments, the non-size increased lymphocytes possess a diameter of 7.0 micrometers or less, 7.5 micrometers or less, or 8.0 micrometers or less.

In certain embodiments, the size-based selection method comprises an array-based method (e.g. a deterministic lateral displacement method). In certain embodiments, the size-based selection method comprises acousticpheresis. In certain embodiments, the size-based selection method comprises a dielectrophoretic method.

In certain embodiments, the array-based separation comprises a microfluidic device configured for deterministic lateral displacement. In certain embodiments, the microfluidic device comprises a plurality of arrays comprising a plurality of obstacles arranged into rows running approximately perpendicular to a direction of fluid flow and columns running approximately parallel to the direction of fluid flow, wherein the columns are offset from the direction of fluid flow by a tilt angle. In certain embodiments, the device comprises at least 50 arrays of obstacles. In certain embodiments, the device comprises at least 50 arrays of obstacles arranged in parallel. In certain embodiments, the plurality of obstacles comprises at least 50 rows of obstacles. In certain embodiments, the plurality of obstacles comprises at least 50 columns of obstacles.

In certain embodiments, the microfluidic device comprises an array of posts having a diameter of about 20 μm. In certain embodiments, a buffer flows continuously through the microfluidic device. In certain embodiments, the microfluidic device operates in oscillatory flow conditions. In certain embodiments, the flow rate through the microfluidic device is at least about 500 mL per hour. In certain embodiments, the flow rate through the microfluidic device is at least about 600 mL per hour. In certain embodiments, the flow rate through the microfluidic device is at least about 700 mL per hour. In certain embodiments, the flow rate through the microfluidic device is at least about 800 mL per hour. In certain embodiments, the flow rate through the microfluidic device is at least about 900 mL per hour. In certain embodiments, the flow rate through the microfluidic device is at least about 1000 mL per hour. In certain embodiments, the microfluidic device comprises an array of asymmetric hexagonal obstacles. In certain embodiments, the microfluidic device comprises a plurality of obstacles having a diamond shape. In certain embodiments, the microfluidic device comprises a plurality of obstacles having a circular or ellipsoid shape. In certain embodiments, each obstacle of the plurality of obstacles has a diamond, circular, ellipsoid, or hexagonal shape. In certain embodiments, each obstacle of plurality of obstacles has a horizontal P1 length approximately parallel to the direction of fluid flow that is longer than a P2 length approximately perpendicular to the direction of fluid flow. In certain embodiments, each obstacle of the plurality of obstacles has an elongated hexagonal shape.

In certain embodiments, P1 is about 10 μm to about 60 μm and P2 is about 10 μm to about 30 μm. In certain embodiments, P1 is about 40 μm and P2 is about 20 μm. In certain embodiments, P1 is 50% to 150% longer than P2. In certain embodiments, the obstacles in a column are separated by a G1 gap of about 22 μm and the obstacles in a row of obstacles are separated by a G2 gap of about 17 μm. In certain embodiments, the obstacles in a column of obstacles are separated by a G1 gap of about 20 to 35 μm and the obstacles in a row of obstacles are separated by a G2 gap of about 15 to 20 μm. In certain embodiments, the obstacles in a column of obstacles are separated by a G1 gap of about 22 to 34 μm and the obstacles in a row of obstacles are separated by a G2 gap of about 16 to 18 μm. In certain embodiments, the obstacles in a column of obstacles are separated by a G1 gap of about 22 μm and the obstacles in a row of obstacles are separated by a G2 gap of about 16 to 18 μm. In certain embodiments, the obstacles in a column of obstacles are separated by a G1 gap of about 26 μm and the obstacles in a row of obstacles are separated by a G2 gap of about 16 to 18 μm.

In certain embodiments, the microfluidic device comprises a plurality of obstacles having vertices that extend into parallel gaps such that the gaps are flanked on either side by one or more vertices pointing toward one another but not directly opposite one another. In certain embodiments, the microfluidic device comprises a plurality of obstacles have vertices that extend into perpendicular gaps such that the gaps are flanked on either side by vertices pointing toward one another and that are directly opposite one another. In certain embodiments, the microfluidic device comprises a plurality of obstacles arranged such that the tilt angle is 1/100. In certain embodiments, the microfluidic device comprises a plurality of obstacles arranged into at least 50 columns. In certain embodiments, the microfluidic device comprises a plurality of obstacles arranged into at least about 50 rows. In certain embodiments, the microfluidic device comprises a first and/or second planar support which comprise at least 20 embedded channels.

In certain embodiments, the microfluidic device comprises (a) a first plurality of arrays comprising a plurality of obstacles arranged into rows running approximately perpendicular to a direction of fluid flow and columns running approximately parallel to the direction of fluid flow, wherein the columns are offset from the direction of fluid flow by a tilt angle; and (b) a second plurality of arrays comprising a plurality of obstacles arranged into rows running approximately perpendicular to a direction of fluid flow and columns running approximately parallel to the direction of fluid flow, wherein the columns are offset from the direction of fluid flow by a tilt angle. In certain embodiments, the obstacles in a column of obstacles of the first plurality of arrays are separated by a G1 gap of about 22 μm and the obstacles in a row of obstacles are separated by a G2 gap of about 16 to 18 μm. In certain embodiments, the obstacles in a column of obstacles of the second plurality of arrays are separated by a G1 gap of about 22 μm and the obstacles in a row of obstacles are separated by a G2 gap of about 16 to 18 μm. In certain embodiments, the obstacles in a column of obstacles of the second plurality of arrays are separated by a G1 gap of about 26 μm and the obstacles in a row of obstacles are separated by a G2 gap of about 16 to 18 μm. In certain embodiments, the obstacles in a column of obstacles of the second plurality of arrays are separated by a G1 gap of about 30 μm and the obstacles in a row of obstacles are separated by a G2 gap of about 16 to 18 μm. In certain embodiments, the obstacles in a column of obstacles of the second plurality of arrays are separated by a G1 gap of about 34 μm and the obstacles in a row of obstacles are separated by a G2 gap of about 16 to 18 μm.

In certain embodiments, the large cells are removed before the small cells are removed. In certain embodiments, the small cells are removed before the large cells are removed. In certain embodiments, the small cells and the large cells are removed simultaneously. In certain embodiments, separating non-size increased lymphocytes from the size increased lymphocytes is performed using a size based selection method. In certain embodiments, separating non-size increased lymphocytes from the size increased lymphocytes occurs at least 4 hours, 8 hours, 12 hours, 24 hours, 36 hours, or 48 hours after contacting the lymphocytes with the size increasing agent. In certain embodiments, separating non-size increased lymphocytes from the size increased lymphocytes occurs prior to 24 hours, 36 hours, 48 hours, or 60 hours after contacting the lymphocytes with the size increasing agent. In certain embodiments, separating the small cells from the biological sample does not use a reagent with affinity for the small cells. In certain embodiments, the reagent with affinity for the small cells is an antibody or antigen binding fragment thereof that binds to a surface marker of the small cells. In certain embodiments, separating the large cells from the biological sample does not use a reagent with affinity for the large cells. In certain embodiments, the reagent with affinity for the large cells is not an antibody or antigen binding fragment thereof that binds to a surface marker of the large cells. In certain embodiments, separating the non-size increased lymphocytes from the size increased lymphocytes does not use a reagent with affinity for the inactivated lymphocytes. In certain embodiments, the reagent with affinity for the large cells is not an antibody or antigen binding fragment thereof that binds to a surface marker of the large cells. In certain embodiments, the non-size increased lymphocytes comprise B cells. In certain embodiments, the non-size increased lymphocytes comprise T cells. In certain embodiments, the non-size increased lymphocytes comprise NK cells. In certain embodiments, the non-size increased lymphocytes comprise NKT cells. In certain embodiments, the enriched size increased lymphocytes comprise B cells, T cells, NK cells, or combinations thereof. In certain embodiments, the enriched size increased lymphocytes comprise B cells. In certain embodiments, the enriched size increased lymphocytes comprise NKT cells. In certain embodiments, the enriched size increased lymphocytes comprise T cells. In certain embodiments, the enriched size increased lymphocytes comprise NK cells. In certain embodiments, the method further comprises genetically engineering the enriched size increased lymphocytes or the size increased lymphocytes to produce genetically engineered size increased lymphocytes or genetically engineered enriched size increased lymphocytes. In certain embodiments, the genetically engineered size increased lymphocytes express a chimeric antigen receptor. In certain embodiments, the genetically engineered size increased lymphocytes are genetically engineered by use of a virus, plasmid DNA, or mRNA.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 illustrates a non-limiting workflow for separation and activation of lymphocytes according to the methods described herein.

FIG. 2 illustrates a non-limiting workflow for separation and activation of T cells according to the methods described herein.

FIG. 3 illustrates the sizes of different blood cells as determined by axial light loss, different patient samples are shown.

FIG. 4 shows % recovery of lymphocytes, granulocytes, or monocytes from leukopaks subjected to different size cut-offs. N=9 for 4.2 micrometers; N=3 for 5.5 micrometers; N=6 for 6.5 micrometers; N=4 for 7.5 micrometers.

FIG. 5 shows net recovery of different cell types subjected to different size cut-offs.

FIG. 6 shows recovery of different cell types following 4.2+ separation then subjected to a 6.5− separation after culture for the specified amount of days.

FIG. 7 shows recovery of different cell types following 4.2+ separation then subjected to a 6.5− separation, after activation, after culture for the specified amount of days.

### DETAILED DESCRIPTION

Described herein is a method of enriching lymphocytes from a biological sample, the method comprising: (a) separating large cells and small cells from the biological sample by a size based selection method to obtain lymphocytes, wherein the large cells comprise granulocytes or monocytes, or a combination thereof, and the small cells comprise platelets, red blood cells, or a combination thereof.

Described herein is a method of enriching lymphocytes from a biological sample, the method comprising: (a) separating large cells and small cells from the biological sample by a size based selection method to obtain lymphocytes, wherein the large cells comprise granulocytes or monocytes, or a combination thereof, and the small cells comprise platelets, red blood cells, or a combination thereof; and (b) contacting the lymphocytes with an activating agent to obtain activated lymphocytes.

Described herein is a method of enriching lymphocytes from a biological sample, the method comprising: (a) separating large cells and small cells from the biological sample by a size based selection method to obtain lymphocytes, wherein the large cells comprise granulocytes or monocytes, or a combination thereof, and the small cells comprise platelets, red blood cells, or a combination thereof; (b) contacting the lymphocytes with an activating agent to obtain activated lymphocytes; and (c) separating inactivated lymphocytes from the activated lymphocytes using a sized based selection method to obtain activated enriched lymphocytes, thereby obtaining enriched activated lymphocytes.

A method of enriching lymphocytes from a biological sample, the method comprising: separating large cells and small cells from the biological sample by a size based selection method to obtain lymphocytes, wherein the large cells comprise granulocytes monocytes, or a combination thereof, and the small cells comprise platelets, red blood cells, or a combination thereof, contacting the lymphocytes with a size increasing agent to obtain size increased lymphocytes; and separating non-size increased lymphocytes from the size increased lymphocytes to obtain size increased enriched lymphocytes, thereby obtaining enriched size increased lymphocytes.

Also described herein are methods of enriching lymphocytes from a biological sample, the method comprising: applying a biological sample to a device configured to separate large cells and small cells from the biological sample by a size based selection method to obtain lymphocytes, wherein the large cells comprise granulocytes monocytes, or a combination thereof, and the small cells comprise platelets, red blood cells, or a combination thereof; contacting the lymphocytes with a size increasing agent to obtain size increased lymphocytes; and applying a population of cells comprising the size increased lymphocytes to a device configured to separate non-size increased lymphocytes from the size increased lymphocytes to obtain size increased enriched lymphocytes, thereby obtaining enriched size increased lymphocytes. In certain embodiments, the device configured to separate large cells and small cells from the biological sample is a DLD device. In certain embodiments, the device configured to separate non-size increased lymphocytes from the size increased lymphocytes is a DLD device.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

As used herein the term “exogenous’ with respect to a nucleic acid or polypeptide refers to a nucleic acid or polypeptide, which has been intentionally introduced into a cell to genetically engineer the cell to alter the function or increase the therapeutic potential of the cell. Exogenous molecules may be derived from various sources and can comprise synthetic or engineered nucleotides or polypeptides, molecules from a different species or cell-type sources compared to the cell that has the exogenous nucleic acid introduced.

The term “genetic engineering” and grammatical equivalents herein refer to the introduction of an exogenous nucleic acid (e.g., a DNA or RNA) into a cell to alter the cells genetic make-up or phenotype and includes, but is not limited to inserting a nucleic acid into or deleting a portion of the cells genome or introducing a nucleic acid encoding an RNA or polypeptide. Cells that are genetically engineered include those that have one or more genetic elements or alterations introduced to the cell, including substitution, deletion, or insertion of one or more nucleotides, or nucleic acids encoding an ectopically expressed polypeptide or RNA. Genetic engineering includes genetic elements that integrate into the genome or that may be maintained extra-genomically (e.g., episomally). Genetic engineering includes the introduction of RNAs that encode polypeptides or catalytic RNA species. Nucleic acids can be introduced into cells as described herein by use of a virus (e.g., adenovirus, lentivirus, adeno-associated virus), a transfection reagent (e.g., cationic lipid polymers), electroporation, or mechanical stress.

The terms “isolate” and “purify” unless otherwise indicated, are synonymous and refer to the enrichment of a desired product relative to a beginning amount. The terms do not necessarily mean that the product is completely isolated or completely pure. For example, if a starting sample had a target cell that constituted 2% of the cells in a sample, and a procedure was performed that resulted in a composition in which the target cell was 60% of the cells present, the procedure would have succeeded in isolating or purifying the target cell.

The term “activating agent” as described herein refers to a chemical substance or biological macromolecule that initiates one or more increases in metabolic activity (e.g., organelle synthesis, protein synthesis, etc.) or cell-division events (DNA synthesis, mitosis, etc.) in a cell to which the activating agent is contacted resulting in an increased size of the cell. Suitable activating agents include receptor or cell surface marker agonistic antibodies, ligands for a receptor, mitogens, peptides, toxins, or other small-molecule cell activators or stressors.

The terms “obstacle array”, “DLD array”, and “array” are used synonymously herein and describe an ordered array of obstacles that are disposed in a flow channel through which a cell or particle-bearing fluid can be passed. An obstacle array comprises a plurality of obstacles arranged in a column (along the path of fluid flow). Gaps between the obstacles (along the path of the fluid flow) allow the passage of cells or other particles. Such obstacles or columns can be arranged into one or more repeating rows (perpendicular to the path of fluid flow).

As described herein a “channel” or “lane” refers to a discreet separation unit with a plurality of obstacles that may be bounded on either side by walls such that discreet lanes are separated. Channels may run in parallel from one or more common inputs to one or more common outputs. Channels may be fluidly connected in series.

As described herein, the terms “fluid flow” and “bulk fluid flow” as used herein in connection with DLD refer to the macroscopic movement of fluid in a general direction across an obstacle array. These terms do not take into account the temporary displacements of fluid streams for fluid to move around an obstacle in order for the fluid to continue to move in the general direction.

As described herein, the term “tilt angle” or “F”: is the angle between the direction of bulk fluid flow and the direction defined by alignment of rows of sequential obstacles in an obstacle array.

As described herein, the term “array direction” is a direction defined by the alignment of rows of sequential obstacles in an obstacle array. A particle is “deflected” or “bumped” in an obstacle array if, upon passing through a gap and encountering a downstream obstacle, the particle's overall trajectory follows the direction of the columns of the obstacle array (i.e., travels at the tilt angle F relative to bulk fluid flow). A particle is not bumped if its overall trajectory follows the direction of bulk fluid flow under those circumstances.

The term “Deterministic Lateral Displacement” or “DLD” refers to a process in which particles are deflected on a path through an array, deterministically, based on their size in relation to some of the array parameters. This process can be used to separate cells, which is generally the context in which it is discussed herein. However, it is important to recognize that DLD can also be used to concentrate cells and for buffer exchange. Processes are generally described herein in terms of continuous flow (DC conditions; i.e., bulk fluid flow in only a single direction). However, DLD can also work under oscillatory flow (AC conditions; i.e., bulk fluid flow alternating between two directions).

The “critical size” or “predetermined size,” “critical diameter” or “predetermined diameter” of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced. When a profile of fluid flow through a gap is symmetrical about the plane that bisects the gap in the direction of bulk fluid flow, the critical size can be identical for both sides of the gap; however, when the profile is asymmetrical, the critical sizes of the two sides of the gap can differ. Arrays can be configured so that cells or particles greater than or less than a critical size may be collected. In some instances, a critical size range may be achieved by combining two or more arrays with their own critical size, and having multiple exits to collect the greater than and less than fractions from each array.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

The polypeptides described herein can be encoded by a nucleic acid. A nucleic acid is a type of polynucleotide (DNA or RNA) comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

As described herein lymphocytes are cells that descend from a common lymphoid progenitor and comprise T cells, B cells, natural killer cells, and natural killer T cells, and exclude cells derived from the myeloid lineage (e.g., monocytes, macrophages, dendritic cells, neutrophils, eosinophils, basophils, mast cells, etc.). Lymphocytes are generally identified as CD45+, and FSC^{dim}, SSC^{dim} when read on a flow cytometer. Activated lymphocytes are those that have encountered immunological signals that have led to the induction of their adaptive immune function, including activation though innate immune mechanisms, cytokines, chemokines, immune rector engagement, etc. and can be assessed by surface markers generally associate with activation.

### Separation of Cells

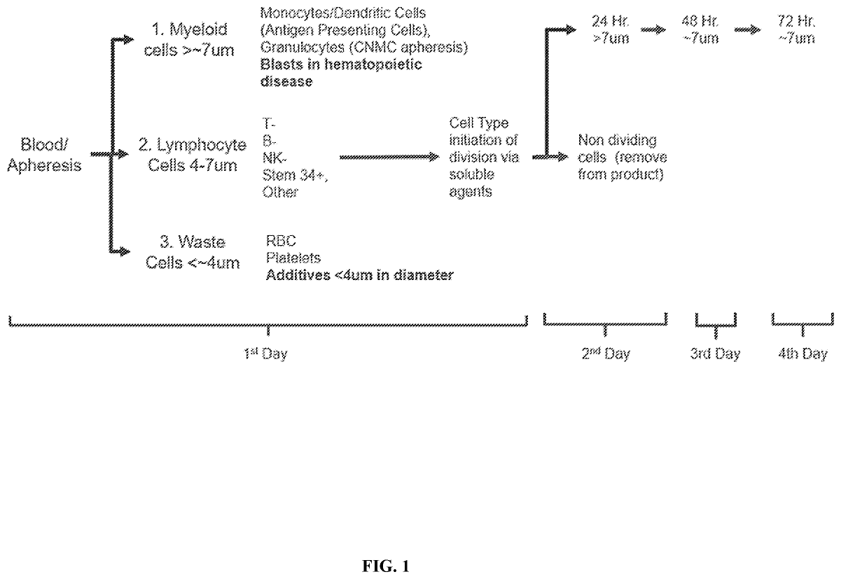

Described herein are methods useful for the isolation, purification, and optionally, the genetic engineering of activated cell populations. Referring to FIG. 1 an exemplary workflow for separation comprises removing cells that are larger and smaller than a particular target cell population from a sample, activating the target cell population, and then applying another size based separation step that isolates the activated cells. In the first step a target cell population comprising T-cells, B-cells, or NK cells, or hematopoietic stem cells can be isolated by removing large cells possessing a diameter in excess of 7 micrometers, and removing small cells possessing a diameter less than about 3.5 or 4 micrometers in diameter. In general, large cells that will be removed by this process comprise granulocytes, macrophages, dendritic cells, monocytes, and blast cells (present in hematopoietic malignancies). In general, small cells will comprise platelets and/or red blood cells. Such separation can occur in series by removing either large cells and then small cells, or by removing small cells followed by large cells; or by simultaneous or overlapping removal of small and large cells. Cells are then activated by an appropriate activating agent causing the cells to increase in size, and subsequently removing the larger activated cells (e.g., cells in excess of 7 microns from the smaller non-activated cells. Cells can be genetically engineered at either the activation step or after the activated cells have been isolated.

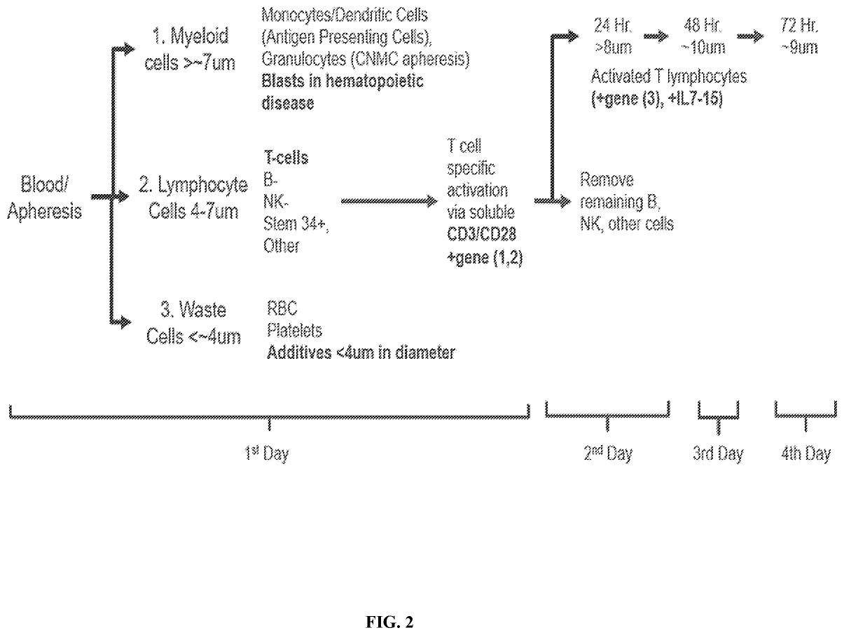

Described herein are methods useful for the isolation and purification and optionally the genetic engineering of activated T-cell populations. Referring to FIG. 2 an exemplary workflow for separation comprises removing cells that are larger and smaller than a particular target cell population from a sample, activating the target cell population, and then applying another size based separation step that isolates the activated cells. In the first step a target cell population comprising T-cells, B-cells, or NK cells, or hematopoietic stem cells can be isolated by removing large cells possessing a diameter in excess of 7 micrometers, and removing small cells possessing a diameter less than about 3.5 or 4 micrometers in diameter. Large cells that will be removed by this process comprise granulocytes, macrophages, dendritic cells, monocytes, and blast cells (present in hematopoietic malignancies). Small cells will comprise platelets and/or red blood cells. Such separation can occur in series by removing either large cells and then small cells, or by removing small cells followed by large cells; or by simultaneous or overlapping removal of small and large cells. Cells are then activated by an appropriate activating agent such as a CD3/CD28 antibody cocktail, causing the cells to grow and divide, and subsequently removing the larger activated cells (e.g., cells in excess of 7 microns from the smaller non-activated cells. Cells can be genetically engineered at either the activation step or after the activated cells have been isolated.

A cell's size can be determined using various measurements sch as microscopy or flow cytometry, however for the purposes of this disclosure the size and/or diameter of a cell is that which is measured by axial light loss (ALL) as described in Downey G P, et al. “Retention of leukocytes in capillaries: role of cell size and deformability.” J Appl Physiol (1985). 1990 November; 69(5):1767-78. Importantly, buffer composition has an influence on sizes with citrated buffers (often used in blood products like a leukapheresis product) shrinking cells by 10 to 15% compared to isotonic pH buffers such as PBS, thus cell size may vary depending on the buffer or diluent in which the cells are suspended prior to separation.

### Samples

The samples form which target cells are isolated, activated can be any biological samples comprising the desired target cells. In certain embodiments, the sample is a sample that comprises at least one or more of a B cell, T cell, NK cell, or a hematopoietic stem cell and one or more platelets, red blood cells, granulocytes, macrophages, monocytes, blast cells, or dendritic cells. In certain embodiments, the biological sample is a blood-related sample. Blood-related samples for use according to the methods described herein include any sample comprising platelets, red blood cells, and one or more additional cell types. Blood-related samples can be whole-blood samples derived from one or more donors. Additional, samples can be those that have been previously subjected to partial or complete apheresis procedures, such as plasmapheresis, plateletpheresis, erythrocytapheresis, or leukapheresis. In certain embodiments, the blood related sample is an apheresis product. In certain embodiments, the blood related sample is a leukapheresis product (also referred to as a leukopak). The blood related sample may comprise a volume in excess of about 1 milliliter, about 2 milliliters, about 5 milliliters, about 10 milliliters, about 25 milliliters, about 50 milliliters, about 100 milliliters, about 250 milliliters, about 500 milliliters, or more (before or after dilution with a compatible diluent). Blood related samples may be collected into buffer, a bag or a coated tube comprising an ant-coagulant such as EDTA, heparin or acid citrate dextrose (ACD). An anti-coagulant such as EDTA, heparin, or ACD, or citrate in the absence of dextrose, may be added to blood related sample after collection. A blood related sample may be stored prior to being subjected to the methods described herein at 8, 6, or 4 degrees or lower for several hours or days.

The blood related sample may comprise a hematocrit of at least 1%, 2%, 4%, 6%, 8%, 10% or higher. The blood related sample may comprise a hematocrit of 60%, 50%, 40%, 30%, 20%, 10% or lower.

### Large Cells

Large cells as described herein may comprise granulocytes, monocytes, macrophages, dendritic cells, blast cells, or combinations thereof. In certain embodiments, large cells comprise cells possessing a diameter of greater than 6.5 micrometers. In certain embodiments, large cells comprise cells possessing a diameter of greater than 7.0 micrometers. In certain embodiments, large cells comprise cells possessing a diameter of greater than 7.5 micrometers. In certain embodiments, large cells comprise cells possessing a diameter of greater than 8.0 micrometers. In certain embodiments, large cells comprise cells possessing a diameter of greater than 8.5 micrometers. In certain embodiments, large cells comprise cells possessing a diameter of greater than 9.0 micrometers. In certain embodiments, large cells comprise cells possessing a diameter of greater than 6.1 micrometers. In certain embodiments, large cells comprise cells possessing a diameter of greater than 6.2 micrometers. In certain embodiments, large cells comprise cells possessing a diameter of greater than 6.3 micrometers. In certain embodiments, large cells comprise cells possessing a diameter of greater than 6.4 micrometers. In certain embodiments, large cells comprise cells possessing a diameter of greater than 6.5 micrometers. In certain embodiments, large cells comprise cells possessing a diameter of greater than 6.6 micrometers. In certain embodiments, large cells comprise cells possessing a diameter of greater than 6.7 micrometers. In certain embodiments, large cells comprise cells possessing a diameter of greater than 6.8 micrometers. In certain embodiments, large cells comprise cells possessing a diameter of greater than 6.9 micrometers.

The methods described herein advantageously remove large cells from a sample. Many larger lymphoid and or myeloid cells exert biological effect on T cells, B cells, NK cells. These effects are pleiotropic and vary depending upon the exact nature of the large cell, but they may result in unwanted activation, suppression of effector function, or premature differentiation. Also blast cells that are often present in the blood of those afflicted with leukemia are desirable to remove as they may have downstream negative effects on any cell therapy produced from leukemia patients blood products (e.g., autologous cell therapies). The methods described herein can remove large cells and reduce their numbers with respect to various target cells (e.g., lymphocytes, T cells, B cells, NK cells) as a reduced ratio of target cells to large cells.

Additionally, the large cells removed such as dendritic cells, monocytes, or macrophages may be collected for use in production of cell therapy compositions. Such cells can be further contacted with an activating agent.

In some embodiments, the ratio of large cells to target cells achieved by the methods described herein is about 0.001:1 to about 0.01:1, about 0.001:1 to about 0.1:1, about 0.001:1 to about 1:1, about 0.001:1 to about 10:1, about 0.001:1 to about 25:1, about 0.001:1 to about 50:1, about 0.001:1 to about 100:1, about 0.001:1 to about 200:1, about 0.001:1 to about 300:1, about 0.001:1 to about 400:1, about 0.001:1 to about 500:1, about 0.01:1 to about 0.1:1, about 0.01:1 to about 1:1, about 0.01:1 to about 10:1, about 0.01:1 to about 25:1, about 0.01:1 to about 50:1, about 0.01:1 to about 100:1, about 0.01:1 to about 200:1, about 0.01:1 to about 300:1, about 0.01:1 to about 400:1, about 0.01:1 to about 500:1, about 0.1:1 to about 1:1, about 0.1:1 to about 10:1, about 0.1:1 to about 25:1, about 0.1:1 to about 50:1, about 0.1:1 to about 100:1, about 0.1:1 to about 200:1, about 0.1:1 to about 300:1, about 0.1:1 to about 400:1, about 0.1:1 to about 500:1, about 1:1 to about 10:1, about 1:1 to about 25:1, about 1:1 to about 50:1, about 1:1 to about 100:1, about 1:1 to about 200:1, about 1:1 to about 300:1, about 1:1 to about 400:1, about 1:1 to about 500:1, about 10:1 to about 25:1, about 10:1 to about 50:1, about 10:1 to about 100:1, about 10:1 to about 200:1, about 10:1 to about 300:1, about 10:1 to about 400:1, about 10:1 to about 500:1, about 25:1 to about 50:1, about 25:1 to about 100:1, about 25:1 to about 200:1, about 25:1 to about 300:1, about 25:1 to about 400:1, about 25:1 to about 500:1, about 50:1 to about 100:1, about 50:1 to about 200:1, about 50:1 to about 300:1, about 50:1 to about 400:1, about 50:1 to about 500:1, about 100:1 to about 200:1, about 100:1 to about 300:1, about 100:1 to about 400:1, about 100:1 to about 500:1, about 200:1 to about 300:1, about 200:1 to about 400:1, about 200:1 to about 500:1, about 300:1 to about 400:1, about 300:1 to about 500:1, or about 400:1 to about 500:1. In some embodiments, the ratio of large cells to target cells is about 0.001:1, about 0.01:1, about 0.1:1, about 1:1, about 10:1, about 25:1, about 50:1, about 100:1, about 200:1, about 300:1, about 400:1, or about 500:1. In some embodiments, the ratio of large cells to target cells at most about 0.01:1, about 0.1:1, about 1:1, about 10:1, about 25:1, about 50:1, about 100:1, about 200:1, about 300:1, about 400:1, or about 500:1. The large cells may be any one or more of dendritic cells, granulocytes, macrophages, or monocytes.

The methods described herein provide lymphocyte populations or enriched activated lymphocytes with reduced amounts of large cells. In certain embodiments, large cells may make up 30%, 25%, 20%, 15%, 10%, or 5% or less of the lymphocyte population after removal. In certain embodiments, large cells may make up 30%, 25%, 20%, 15%, 10%, or 5% or less of the enriched activated lymphocytes after removal. In certain embodiments, large cells may be reduced in number from a starting number in a biological sample by 50%, 60%, 70%, 75%, 80%, 85%, 90%, or 95% or greater. In certain embodiments, the large cells comprise granulocytes, dendritic cells, monocytes, macrophages, blast cells, or any combination thereof.

### Small Cells

The methods described herein remove small cells and/or vesicles. Small cells or vesicles as described herein may comprise red blood cells, platelet cells, or vesicles derived from cells including platelet microparticles, microvesicles, exosomes, or a combination thereof. In certain embodiments, small cells or vesicles comprise cells or vesicles possessing a diameter or critical size of less than 5.5 micrometers. In certain embodiments, small cells or vesicles comprise cells or vesicles possessing a diameter of less than 5 micrometers. In certain embodiments, small cells or vesicles comprise cells or vesicles possessing a diameter of less than 4.5 micrometers. In certain embodiments, small cells or vesicles comprise cells or vesicles possessing a diameter of less than 4.4 micrometers. In certain embodiments, small cells or vesicles comprise cells or vesicles possessing a diameter of less than 4.3 micrometers. In certain embodiments, small cells or vesicles comprise cells or vesicles possessing a diameter of less than 4.2 micrometers. In certain embodiments, small cells or vesicles comprise cells or vesicles possessing a diameter of less than 4.1 micrometers. In certain embodiments, small cells or vesicles comprise cells or vesicles possessing a diameter of less than 4 micrometers. In certain embodiments, small cells or vesicles comprise cells or vesicles possessing a diameter of less than 3.5 micrometers. In certain embodiments, small cells or vesicles comprise cells or vesicles possessing a diameter of less than 3 micrometers. In certain embodiments, small cells or vesicles comprise cells or vesicles possessing a diameter of less than 2 micrometers. In certain embodiments, small cells or vesicles comprise cells or vesicles possessing a diameter of less than 1 micrometers.

The methods described herein advantageously remove platelet cells and platelet microparticles and soluble factors, such as cytokines or chemokines, which may contribute to unwanted cell activation due to the release of pro-thrombic factors and other factors which may influence lymphocyte activation and/or function. The methods described herein can remove platelet cells and reduce their numbers with respect to various target cells (e.g., lymphocytes, T cells, B cells, NK cells, hematopoietic stem cells) as a reduced ratio of target cells to platelets.

In some embodiments, the ratio of platelets to target cells achieved by the methods described herein is about 0.001:1 to about 0.01:1, about 0.001:1 to about 0.1:1, about 0.001:1 to about 1:1, about 0.001:1 to about 10:1, about 0.001:1 to about 25:1, about 0.001:1 to about 50:1, about 0.001:1 to about 100:1, about 0.001:1 to about 200:1, about 0.001:1 to about 300:1, about 0.001:1 to about 400:1, about 0.001:1 to about 500:1, about 0.01:1 to about 0.1:1, about 0.01:1 to about 1:1, about 0.01:1 to about 10:1, about 0.01:1 to about 25:1, about 0.01:1 to about 50:1, about 0.01:1 to about 100:1, about 0.01:1 to about 200:1, about 0.01:1 to about 300:1, about 0.01:1 to about 400:1, about 0.01:1 to about 500:1, about 0.1:1 to about 1:1, about 0.1:1 to about 10:1, about 0.1:1 to about 25:1, about 0.1:1 to about 50:1, about 0.1:1 to about 100:1, about 0.1:1 to about 200:1, about 0.1:1 to about 300:1, about 0.1:1 to about 400:1, about 0.1:1 to about 500:1, about 1:1 to about 10:1, about 1:1 to about 25:1, about 1:1 to about 50:1, about 1:1 to about 100:1, about 1:1 to about 200:1, about 1:1 to about 300:1, about 1:1 to about 400:1, about 1:1 to about 500:1, about 10:1 to about 25:1, about 10:1 to about 50:1, about 10:1 to about 100:1, about 10:1 to about 200:1, about 10:1 to about 300:1, about 10:1 to about 400:1, about 10:1 to about 500:1, about 25:1 to about 50:1, about 25:1 to about 100:1, about 25:1 to about 200:1, about 25:1 to about 300:1, about 25:1 to about 400:1, about 25:1 to about 500:1, about 50:1 to about 100:1, about 50:1 to about 200:1, about 50:1 to about 300:1, about 50:1 to about 400:1, about 50:1 to about 500:1, about 100:1 to about 200:1, about 100:1 to about 300:1, about 100:1 to about 400:1, about 100:1 to about 500:1, about 200:1 to about 300:1, about 200:1 to about 400:1, about 200:1 to about 500:1, about 300:1 to about 400:1, about 300:1 to about 500:1, or about 400:1 to about 500:1. In some embodiments, the ratio of platelets to target cells is about 0.001:1, about 0.01:1, about 0.1:1, about 1:1, about 10:1, about 25:1, about 50:1, about 100:1, about 200:1, about 300:1, about 400:1, or about 500:1. In some embodiments, the ratio of platelets to target cells at most about 0.01:1, about 0.1:1, about 1:1, about 10:1, about 25:1, about 50:1, about 100:1, about 200:1, about 300:1, about 400:1, or about 500:1.

The methods described herein can also alternatively or in addition remove red blood cells (RBCs). The methods described herein can remove RBCs and reduce their numbers with respect to various target cells (e.g., lymphocytes, T cells, B cells, NK cells, hematopoietic stem cells) as a reduced ratio of target cells to RBCs

In some embodiments, the ratio of RBCs to target cells achieved by the methods described herein is about 0.001:1 to about 0.01:1, about 0.001:1 to about 0.1:1, about 0.001:1 to about 1:1, about 0.001:1 to about 10:1, about 0.001:1 to about 25:1, about 0.001:1 to about 50:1, about 0.001:1 to about 100:1, about 0.001:1 to about 200:1, about 0.001:1 to about 300:1, about 0.001:1 to about 400:1, about 0.001:1 to about 500:1, about 0.01:1 to about 0.1:1, about 0.01:1 to about 1:1, about 0.01:1 to about 10:1, about 0.01:1 to about 25:1, about 0.01:1 to about 50:1, about 0.01:1 to about 100:1, about 0.01:1 to about 200:1, about 0.01:1 to about 300:1, about 0.01:1 to about 400:1, about 0.01:1 to about 500:1, about 0.1:1 to about 1:1, about 0.1:1 to about 10:1, about 0.1:1 to about 25:1, about 0.1:1 to about 50:1, about 0.1:1 to about 100:1, about 0.1:1 to about 200:1, about 0.1:1 to about 300:1, about 0.1:1 to about 400:1, about 0.1:1 to about 500:1, about 1:1 to about 10:1, about 1:1 to about 25:1, about 1:1 to about 50:1, about 1:1 to about 100:1, about 1:1 to about 200:1, about 1:1 to about 300:1, about 1:1 to about 400:1, about 1:1 to about 500:1, about 10:1 to about 25:1, about 10:1 to about 50:1, about 10:1 to about 100:1, about 10:1 to about 200:1, about 10:1 to about 300:1, about 10:1 to about 400:1, about 10:1 to about 500:1, about 25:1 to about 50:1, about 25:1 to about 100:1, about 25:1 to about 200:1, about 25:1 to about 300:1, about 25:1 to about 400:1, about 25:1 to about 500:1, about 50:1 to about 100:1, about 50:1 to about 200:1, about 50:1 to about 300:1, about 50:1 to about 400:1, about 50:1 to about 500:1, about 100:1 to about 200:1, about 100:1 to about 300:1, about 100:1 to about 400:1, about 100:1 to about 500:1, about 200:1 to about 300:1, about 200:1 to about 400:1, about 200:1 to about 500:1, about 300:1 to about 400:1, about 300:1 to about 500:1, or about 400:1 to about 500:1. In some embodiments, the ratio of RBCs to target cells is about 0.001:1, about 0.01:1, about 0.1:1, about 1:1, about 10:1, about 25:1, about 50:1, about 100:1, about 200:1, about 300:1, about 400:1, or about 500:1. In some embodiments, the ratio of RBCs to target cells at most about 0.01:1, about 0.1:1, about 1:1, about 10:1, about 25:1, about 50:1, about 100:1, about 200:1, about 300:1, about 400:1, or about 500:1.

The methods described herein provide lymphocyte populations or enriched activated lymphocytes with reduced amounts of small cells. In certain embodiments, small cells may make up 30%, 25%, 20%, 15%, 10%, or 5% or less of the lymphocyte population after removal. In certain embodiments, small cells may make up 30%, 25%, 20%, 15%, 10%, or 5% or less of the enriched activated lymphocytes after removal. In certain embodiments, small cells may be reduced in number from a starting number in a biological sample by 50%, 60%, 70%, 75%, 80%, 85%, 90%, or 95% or greater. In certain embodiments, the small cells comprise red blood cells, platelets, extracellular vesicles, or any combination thereof.

### Target Cells

The methods herein in the first step of separation is designed to isolate and enrich certain target cell populations by removal of undesired large or small cells. In certain embodiments, the target cell population comprises lymphocytes. In certain embodiments, the target cell population comprises T cells (CD3+ cells). In certain embodiments, the target cell population comprises B cells (CD19+ cells). In certain embodiments, the target cell population comprises NK cells (CD56+ cells). In certain embodiments, the target cell population comprises hematopoietic stem cells (CD34+ cells). In certain embodiments, the target cell population comprises erythroid precursor cells (CD235+CD71+ cells).

In some embodiments, target cells possess a dimeter of about 3 micrometers to about 8 micrometers. In some embodiments, target cells possess a dimeter of about 3 micrometers to about 3.5 micrometers, about 3 micrometers to about 4 micrometers, about 3 micrometers to about 4.5 micrometers, about 3 micrometers to about 5 micrometers, about 3 micrometers to about 6 micrometers, about 3 micrometers to about 6.5 micrometers, about 3 micrometers to about 7 micrometers, about 3 micrometers to about 7.5 micrometers, about 3 micrometers to about 8 micrometers, about 3.5 micrometers to about 4 micrometers, about 3.5 micrometers to about 4.5 micrometers, about 3.5 micrometers to about 5 micrometers, about 3.5 micrometers to about 6 micrometers, about 3.5 micrometers to about 6.5 micrometers, about 3.5 micrometers to about 7 micrometers, about 3.5 micrometers to about 7.5 micrometers, about 3.5 micrometers to about 8 micrometers, about 4 micrometers to about 4.5 micrometers, about 4 micrometers to about 5 micrometers, about 4 micrometers to about 6 micrometers, about 4 micrometers to about 6.5 micrometers, about 4 micrometers to about 7 micrometers, about 4 micrometers to about 7.5 micrometers, about 4 micrometers to about 8 micrometers, about 4.5 micrometers to about 5 micrometers, about 4.5 micrometers to about 6 micrometers, about 4.5 micrometers to about 6.5 micrometers, about 4.5 micrometers to about 7 micrometers, about 4.5 micrometers to about 7.5 micrometers, about 4.5 micrometers to about 8 micrometers, about 5 micrometers to about 6 micrometers, about 5 micrometers to about 6.5 micrometers, about 5 micrometers to about 7 micrometers, about 5 micrometers to about 7.5 micrometers, about 5 micrometers to about 8 micrometers, about 6 micrometers to about 6.5 micrometers, about 6 micrometers to about 7 micrometers, about 6 micrometers to about 7.5 micrometers, about 6 micrometers to about 8 micrometers, about 6.5 micrometers to about 7 micrometers, about 6.5 micrometers to about 7.5 micrometers, about 6.5 micrometers to about 8 micrometers, about 7 micrometers to about 7.5 micrometers, about 7 micrometers to about 8 micrometers, or about 7.5 micrometers to about 8 micrometers. In some embodiments, target cells possess a dimeter of about 3 micrometers, about 3.5 micrometers, about 4 micrometers, about 4.5 micrometers, about 5 micrometers, about 6 micrometers, about 6.5 micrometers, about 7 micrometers, about 7.5 micrometers, or about 8 micrometers. In some embodiments, target cells possess a dimeter of at least about 3 micrometers, about 3.5 micrometers, about 4 micrometers, about 4.5 micrometers, about 5 micrometers, about 6 micrometers, about 6.5 micrometers, about 7 micrometers, or about 7.5 micrometers. In some embodiments, target cells possess a dimeter of at most about 3.5 micrometers, about 4 micrometers, about 4.5 micrometers, about 5 micrometers, about 6 micrometers, about 6.5 micrometers, about 7 micrometers, about 7.5 micrometers, or about 8 micrometers.

The enriched target cells can represent a greater percentage of cells then they would normally represent in an un enriched or isolated sample. In certain embodiments, lymphocytes may exhibit 2-fold, 3-fold, 4-fold, 5-fold, 10-fold enrichment compared to whole-blood or leukapheresis starting sample. In certain embodiments, T cells may exhibit a 2-fold, 3-fold, 4-fold, 5-fold, 10-fold enrichment compared to whole-blood or leukapheresis starting sample. In certain embodiments, B cells may exhibit a 2-fold, 3-fold, 4-fold, 5-fold, 10-fold enrichment compared to whole-blood or leukapheresis starting sample. In certain embodiments, NK cells may exhibit a 2-fold, 3-fold, 4-fold, 5-fold, 10-fold enrichment compared to whole-blood or leukapheresis starting sample. In certain embodiments, NKT cells may exhibit a 2-fold, 3-fold, 4-fold, 5-fold, 10-fold enrichment compared to whole-blood or leukapheresis starting sample.

The enriched target cells can result in net recovery from the biological sample of at least about 30%, 40%, 50%, 60%, 70%, 80%, 90% or greater compared to the amount present in the biological sample before enrichment. For example, lymphocyte recovery can be at least about 30%, 40%, 50%, 60%, 70%, 80%, 90% or greater compared to the amount of lymphocytes present in the biological sample before enrichment. For example, T cell recovery can be at least about 30%, 40%, 50%, 60%, 70%, 80%, 90% or greater compared to the amount of T cells present in the biological sample before enrichment.

The enriched target cells can result in percentage of cells that is at least 30%, 40%, 50%, 60%, 70%, 80%, 90% or greater compared to the total amount of enriched cells (e.g., after removal of small and large cells). For example, lymphocytes can make up at least 30%, 40%, 50%, 60%, 70%, 80%, 90% or greater of the cells compared to the total amount of enriched cells (e.g., after removal of small and large cells). For example, T cells can make up at least 30%, 40%, 50%, 60%, 70%, 80%, 90% or greater of the cells compared to the total amount of enriched cells (e.g., after removal of small and large cells).

The methods described herein can produce high amounts of enriched target cells from a single biological sample (e.g. a leukopak). In certain embodiments, at least 1×10⁷, 2×10⁷, 2×10⁷, 1×10⁸, 2×10⁸, 5×10⁸, 1×10⁹ or 2×10⁹ enriched target cells can be obtained from a single biological sample (e.g., a leukopak). In certain embodiments, at least 1×10⁷, 2×10⁷, 2×10⁷, 1×10⁸, 2×10⁸, 5×10⁸, 1×10⁹, 2×10⁹3×10⁹, 4×10⁹, 5×10⁹, 6×10⁹, 7×10⁹, 8×10⁹, 9×10⁹, enriched lymphocytes can be obtained from a single biological sample (e.g., a leukopak). In certain embodiments, at least 1×10⁷, 2×10⁷, 2×10⁷, 1×10⁸, 2×10⁸, 5×10⁸, 1×10⁹, 2×10⁹ 3×10⁹, 4×10⁹, 5×10⁹, 6×10⁹, 7×10⁹, 8×10⁹, 9×10⁹, enriched T cells can be obtained from a single biological sample (e.g., a leukopak). Such numbers can be isolated from a leukopak containing at least 50 mL, 100 mL, 200 mL, 300 mL, 400 mL, or 500 mL of apheresis product.

### Activation of Cells

The methods described herein include an activation step or a step that allows increases in size (e.g., growth factors, hydrogels, etc.). Activation allows for cells to grow and divide allowing for their size based separation. The advantages of activation are at least that: 1) the activation allows for the induction of cell division, which further allows for ease of genetic engineering and expression of an exogenous gene or nucleic acid introduced into the cell; and 2) it allows for the separation of desired cells. For example, a mixed lymphocyte population comprising T cells and B cells between 3.5 and 8.5 microns can be activated by a T cell activating agent (e.g., CD28/CD3 antibody cocktail) having primary activity on T cells allowing the T cells to be enriched after activation while the undesired B cells are discarded.

The activation step can occur after 1, 2, 3, 4, 5, 6, 7 or more days following after the lymphocyte enrichment step. The activation step can occur immediately following (e.g., within the first day) following the enrichment step. The activation step can occur within 2 days following the enrichment step. The activation step can occur within 3 days following the enrichment step. The activation step can occur within 4 days following the enrichment step. The activation step can occur within 5 days following the enrichment step. The activation step can occur within 6 days following the enrichment step. The activation step can occur within 7 days following the enrichment step.

The timing of separation subsequent to activation can be important as it allows proper discrimination of cells by size as the cells are allowed to grow and divide. Cells can be subjected subsequent separation step after allowing for at least 8 hours, 12 hours, 16 hours, 20 hours, 24 hours, 36 hours, 48 hours, 60 hours after contacting the cells with an activating agent. Cells can be separated before about 12 hours, 16 hours, 20 hours, 24 hours, 36 hours, 48 hours, 60 hours after contacting the cells with an activating agent.

Activation agents are primarily directed to specific types of desired target cells. While a certain activating agent may act primarily on a specific target cell (e.g., through receptor ligand interaction, agonism by a receptor binding antibody, specific receptor agonists) the effect of the activating agent may lead to some bystander activation of other cell-types. This is especially the case with immune cells which may secrete inflammatory mediators that activate other immune cells. However, such activation can generally be distinguished by time (i.e., primary activation will proceed prior to bystander activation) or magnitude In certain embodiments, the activating agent is one that primarily activates a T cell. In certain embodiments, the activating agent is one that primarily activates a B cell. In certain embodiments, the activating agent is one that primarily activates a NK cell. In certain embodiments, the activating agent is one that primarily activates a gamma-delta T cell. Examples of activation agents and the types of cells activated are further described in Table 1 below.

TABLE 1
Activating agents

| | | | |
|---|---|---|---|
| | | | Size Based |
| | Cell Specific Activation Scheme: | Cell | Selection |
| | Antibodies, hydrogels, or other | Specific | Criteria |
| PBMC Cell/Type | affinity agents activating cells by | Growth | (after |
| Fraction | sny of these moieties: | Factor | activation) |

Pass 1-Large Cells (Dc > ~7 um)

| | | |
|---|---|---|
| Monocytes, | Target/Tumor specific peptides, | N/A |
| | Chimeric Structures (MCH + | |
| | Peptides), hLF1-11, | |
| | Lipopolysaccaride (LPS) | |
| Disease Cells | N/A already activated by | N/A |
| “Blast” cells | disease | |
| Large tumor cells | | |

Pass 2-Lymphoid Cells (Dc > 4 to ~7 um

| | | | |
|---|---|---|---|
| T-Cells (all) | CD3/CD28; (non-surface | IL-7+-15, | >~7-8 um |
| CD3+ | antigen modulating); CD3/ | (IL-2), | |
| | CD49d, Concanavalin-A, | IL-15, | |
| | | IL-21, | |
| | | IL-35 | |
| NK and T-Cells | CD3/CD28/CD2 | (IL-2), | >~7-8 um |
| (NK + T+ | | IL7 | |
| NK and T-Cells | CD28/CD49d | (IL-2), | >~7-8 um |
| (T+ except Treg) | | | |
| NK-Cells | CD335(NKp46)/CD2, Bacteria, | IL-15, | >~7-8 um |
| | Peptido-glycan, LPS, | IL-2 + | |
| | MicroRNA: mlR-150 | OKT-3 | |
| T-Cells | Vd1: PHA, CD137L, | Zol + | >~6-9 um |
| “gamma/delta” | Biphosphonate, BrHPP, HMBPP, | (IL-2), | |
| cells | Zoledronate/Zoldenronic Acid | Zol + | |
| | (Zometa), Bispecific | (IL-2) + | |
| | Antibodies, Anti-BTN3A1 | OKT3 | |
| | Antibody, PHA | (stage 2) | |
| | | IL-15, | |
| | | IL-18 | |
| B-Cells | IgM/IgD, Cross-linked CD154/ | IL-4, | >~6-9 |
| | 40L multimer, Peptides, cpg- | IL-2, | um(?) |
| | Oligonucleotides, R848, other | IL-21 | |
| | Toll Receptor 7-9 agonists, | (Ig+) | |
| | Anti-BCR, CD79a/b, | | |
| | Pervandadate | | |

In certain embodiments, the activating agent that primarily activates a T cell is selected from a CD3 antibody, a CD28 antibody; a CD49d antibody, Concanavalin-A, and combinations thereof. In certain embodiments, the activating agent that primarily activates a B cell is selected from a IgM antibody, an IgD antibody, a CD154/40L multimer, antigen specific peptides, a stimulatory CpG oligonucleotide, R848, a Toll Receptor 7 agonist, a Toll Receptor 9 agonist, an anti-BCR antibody, a CD79 antibody, Pervanadate, and combinations thereof. In certain embodiments, the activating agent that primarily activates an NK cell is selected from aCD335(NKp46) antibody, a CD2 antibody, a CD16 antibody, a TLR2 agonist, Peptido-glycan, lipopolysaccharide, MicroRNA: mIR-150, and combinations thereof. In certain embodiments, the activating agent that primarily activates a gamma-delta T cell is selected from a Vd1 antibody, PHA, CD137L, Biphosphonate, BrHPP, HMBPP, Zoledronate/Zoldenronic Acid (Zometa), -BTN3A1 Antibody, and combinations thereof.

Additionally, cells-specific growth factors, cytokines, and chemokines (such as those listed in Table 1) can activate certain cell types and be included in the activation steps described.

Also contemplated are activation of hybrid populations of target cells for activation such as NK cells and T cells.

### Separation of Activated Cells

According to the methods described herein after large and small cells are removed from a sample the target cells may be activated prior to a subsequent separation step. This subsequent separation step is to remove the activated cells from cells that have been not activated or only partially activated from the first series of separations and activation. Activation allows cells to grow and divide making them susceptible to size discrimination. Activation also allows for genetic engineering and the therapeutic benefits of the activated cells.

In certain embodiments, activated cells are subjected to a subsequent separation step after allowing for at least 4 hours, 8 hours, 12 hours, 16 hours, 20 hours, 24 hours, 36 hours, 48 hours, 60 hours after contacting the cells with an activating agent. In certain embodiments, activated cells are subjected to a subsequent separation step before about 8 hours, 12 hours, 16 hours, 20 hours, 24 hours, 36 hours, 48 hours, 60 hours after contacting the cells with an activating agent.

The subsequent post-activation cell separation can be applied to activated target cells to enrich cells that are larger than the size of the non-activated cell. In certain embodiments, activated cells comprise cells possessing a diameter of greater than 6.0 micrometers. In certain embodiments, activated cells comprise cells possessing a diameter of greater than 6.5 micrometers. In certain embodiments, activated cells comprise cells possessing a diameter of greater than 7.0 micrometers. In certain embodiments, activated cells comprise cells possessing a diameter of greater than 7.5 micrometers. In certain embodiments, activated cells comprise cells possessing a diameter of greater than 8.0 micrometers. In certain embodiments, activated cells comprise cells possessing a diameter of greater than 8.5 micrometers. In certain embodiments, activated cells comprise cells possessing a diameter of greater than 9.0 micrometers.

The activated target cells can comprise B cells, T cells, NK cells, or hematopoietic stem cells. After activation and separation, the activated target cells can comprise at least 60%, 70%, 75%, 80%, 85%, 90%, 95%, 97%, 98%, or 99% of the activated target cell. If genetic engineering is conducted at the activation step or before subsequent separation of the activated cells the cells may be at least 20%, 30%, 40% 50% 60%, 70%, 75%, 80%, 85%, 90%, 95%, 97%, 98%, or 99% positive for the genetic engineering. Such positivity can be assessed by flow-cytometry tracking expression of a fluorescent gene (e.g., EGFP). Such positivity can be either in the sample with target cells themselves intended for therapeutic or experimental use or a side by side control subjected to the same procedures as the cells intended for therapeutic or experimental use.

The activated target cells can result in net recovery from the biological sample of at least about 30%, 40%, 50%, 60%, 70%, 80%, 90% or greater compared to the amount of the non-activated version of the activated target cell present in the biological sample before enrichment. For example, activated lymphocyte recovery can be at least about 300%, 40%, 50%, 60%, 70%, 80%, 90% or greater compared to the amount of lymphocytes present in the biological sample before enrichment. For example, activated T cell recovery can be at least about 30%, 40%, 50%, 60%, 70%, 80%, 90% or greater compared to the amount of T cells present in the biological sample before enrichment. Activated target cells can be generally identified by surface markers using flow cytometry with an antibody for the surface marker compared to an isotype control. For T cells common activation markers comprise CD25, CD71, or costimulatory markers (e.g., CD26, CD27, CD28, CD30, CD154 or CD40L, and CD134). Any of these markers can be used to analyze T cell activation.

The activated target cells can result in percentage of cells that is at least 30%, 40%, 50%, 60%, 70%, 80%, 90% or greater compared to the total amount of enriched cells (e.g., after removal of small and large cells). For example, activated lymphocytes can make up at least 30%, 40%, 50%, 60%, 70%, 80%, 90% or greater of the cells compared to the total amount of enriched cells (e.g., after removal of small and large cells). For example, activated T cells can make up at least 30%, 40%, 50%, 60%, 70%, 80%, 90% or greater of the cells compared to the total amount of enriched cells (e.g., after removal of small and large cells).

The methods described herein can produce high amounts of activated target cells from a single biological sample (e.g. a leukopak). In certain embodiments, at least 1×10⁷, 2×10⁷, 2×10⁷, 1×10⁸, 2×10⁸, 5×10⁸, 1×10⁹ or 2×10⁹ activated target cells can be obtained from a single biological sample (e.g., a leukopak). In certain embodiments, at least 1×10⁷, 2×10⁷, 2×10⁷, 1×10⁸, 2×10⁸, 5×10⁸, 1×10⁹ or 2×10⁹ activated lymphocytes can be obtained from a single biological sample (e.g., a leukopak). In certain embodiments, at least 1×10⁷, 2×10⁷, 2×10⁷, 1×10⁸, 2×10⁸, 5×10⁸, 1×10⁹ or 2×10⁹ activated T cells can be obtained from a single biological sample (e.g., a leukopak).

The methods described herein can produce high amounts of genetically engineered target cells from a single biological sample (e.g. a leukopak). In certain embodiments, at least 1×10⁷, 2×10⁷, 2×10⁷, 1×10⁸, 2×10⁸, 5×10⁸, 1×10⁹ or 2×10⁹ genetically engineered target cells can be obtained from a single biological sample (e.g., a leukopak). In certain embodiments, at least 1×10⁷, 2×10⁷, 2×10⁷, 1×10⁸, 2×10⁸, 5×10⁸, 1×10⁹ or 2×10⁹ genetically engineered lymphocytes can be obtained from a single biological sample (e.g., a leukopak). In certain embodiments, at least 1×10⁷, 2×10⁷, 2×10⁷, 1×10⁸, 2×10⁸, 5×10⁸, 1×10⁹ or 2×10⁹ genetically engineered T cells can be obtained from a single biological sample (e.g., a leukopak).

In some instances a size increasing agent may be added to further increase the size of a non-target cell. This could comprise for example an affinity reagent (such as an antibody) conjugated to a bead that increases the size of the non-target cell. with specificity to a cell surface marker of a non-target cell added before, during, or after the activation step. In certain embodiments, the non-target cell is a B cell, NK cell, NK T cell, or combination thereof. In certain embodiments, an antibody conjugated to a size increasing agent that specifically binds to a cell surface marker of a B cell, NK cell, NK T cell, or combination thereof is added before, during, or after an activation step. In certain embodiments, an antibody conjugated to a size increasing agent that specifically binds to a cell surface marker of a B cell, NK cell, NK T cell, or combination thereof is added before an activation step. In certain embodiments, an antibody conjugated to a size increasing agent that specifically binds to a cell surface marker of a B cell, NK cell, NK T cell, or combination thereof is added during an activation step. In certain embodiments, an antibody conjugated to a size increasing agent that specifically binds to a cell surface marker of a B cell, NK cell, NK T cell, or combination thereof is added after an activation step. In certain embodiments, the size increasing agent is a biocompatible bead. The biocompatible bead can be a polymer, polystyrene, polyvinyl acetate, polyethylene, glass, agarose, alginate gel, or a combination thereof. In certain embodiments, the B cell marker is CD19 or CD20. In certain embodiments, the NK cell marker is CD56.

### Genetic Engineering

The methods described herein are useful for the genetic engineering of a population of target cells that have been removed from a large and a small cell population. In certain embodiments, the methods described herein are useful for genetic engineering of an activated lymphocyte population. In certain embodiments, the methods described herein are useful for genetic engineering of an activated T cell population. In certain embodiments, the methods described herein are useful for genetic engineering of an activated B cell population. In certain embodiments, the methods described herein are useful for genetic engineering of an activated NK cell population.

The cells can either be engineered during the activation step before the size based separation of the activated cells or after the size based separation of the activated cells. Genetic engineering can be conducted by various known technologies, such as, by transduction with a virus, electroporation, a cationic polymer, a lipid based transfection reagent, or combinations thereof. Cationic lipids or polymers comprise, for example PEI, PLL, PAA, PAE, PDMAEMA, DSPC, PEG2000 PE, DOSPA, DOPE, DLin-MC3-DMA, (+)-N,N [bis (2-hydroxyethyl)]-N-methyl-N-[2,3-di(tetradecanoyloxy)propyl]ammonium iodide, or a combination thereof.

In certain embodiments, genetic engineering comprises contacting a target cell population with a lentivirus carrying an exogenous nucleic acid. In certain embodiments, genetic engineering comprises contacting a target cell population with an adenovirus carrying an exogenous nucleic acid. In certain embodiments, genetic engineering comprises contacting a target cell population with an adeno-associated virus carrying an exogenous nucleic acid. In certain embodiments, genetic engineering comprises contacting to an exogenous nucleic acid while an electric current is applied to the cells or after an electric current is applied to the cells. In certain embodiments, genetic engineering comprises contacting a target cell population with a cationic polymer complexed to an exogenous nucleic acid. In certain embodiments, genetic engineering comprises contacting a target cell population with a cationic-lipid complexed to an exogenous nucleic acid.

In certain embodiments, genetic engineering comprises contacting a T cell population with a lentivirus carrying an exogenous nucleic acid. In certain embodiments, genetic engineering comprises contacting a target cell population with an adenovirus carrying an exogenous nucleic acid. In certain embodiments, genetic engineering comprises contacting a T cell population with an adeno-associated virus carrying an exogenous nucleic acid. In certain embodiments, genetic engineering comprises contacting to an exogenous nucleic acid while an electric current is applied to the cells or after an electric current is applied to the cells. In certain embodiments, genetic engineering comprises contacting a T cell population with a cationic polymer complexed to an exogenous nucleic acid. In certain embodiments, genetic engineering comprises contacting a T cell population with a cationic-lipid complexed to an exogenous nucleic acid.

In certain embodiments, genetic engineering comprises contacting a NK cell population with a lentivirus carrying an exogenous nucleic acid. In certain embodiments, genetic engineering comprises contacting a target cell population with an adenovirus carrying an exogenous nucleic acid. In certain embodiments, genetic engineering comprises contacting a NK cell population with an adeno-associated virus carrying an exogenous nucleic acid. In certain embodiments, genetic engineering comprises contacting to an exogenous nucleic acid while an electric current is applied to the cells or after an electric current is applied to the cells. In certain embodiments, genetic engineering comprises contacting a NK cell population with a cationic polymer complexed to an exogenous nucleic acid. In certain embodiments, genetic engineering comprises contacting a NK cell population with a cationic-lipid complexed to an exogenous nucleic acid.

In certain embodiments, genetic engineering comprises contacting a B cell population with a lentivirus carrying an exogenous nucleic acid. In certain embodiments, genetic engineering comprises contacting a target cell population with an adenovirus carrying an exogenous nucleic acid. In certain embodiments, genetic engineering comprises contacting a B cell population with an adeno-associated virus carrying an exogenous nucleic acid. In certain embodiments, genetic engineering comprises contacting to an exogenous nucleic acid while an electric current is applied to the cells or after an electric current is applied to the cells. In certain embodiments, genetic engineering comprises contacting a B cell population with a cationic polymer complexed to an exogenous nucleic acid. In certain embodiments, genetic engineering comprises contacting a B cell population with a cationic-lipid complexed to an exogenous nucleic acid.

The methods described herein can deliver exogenous genes or nucleic acids to a target cell population. The exogenous nucleic acids can comprise an open reading frame coding for a therapeutic protein. The therapeutic protein could be a chimeric antigen receptor (CAR), a recombinant T cell receptor or receptors, a cytokine, a chemokine, an antibody or any combination thereof. The therapeutic protein may be a CAR that can be used to modify a T cell or an NK cell. IN certain embodiments the CAR is one that targets CD19, CD20, is dual specificity for CD19/20 or CD19/22, CD22, CD38, BCMA, CD123, CLL-1, WT-1, CD229, GD2, CD70, CSPG4, CD2, CD7, CD13, CD33, CD117, CEA, Alpha Fetoprotein, Her2/Neu, PSMA, GPC2, GPC3, IDH1, MUC-1, NYESO1, EphA2, EGFRvIII, KRAS (modified TCR), EBV, MSLN (Mesothelin), or a combination thereof.

Genetically, engineered cells also include the introduction of nucleic acids that alter the biology of the cell through gene editing (e.g., CDRISPR/Cas9, TALEN, or homologous recombination). In particular modifications may be made to delete genes that may be undesired in a clinical setting such as an endogenous T cell receptor, an HLA molecule, or an inhibitory molecule (e.g., checkpoint inhibitor molecules).

While cells may be genetically engineered to achieve a certain phenotype in certain cases therapeutically useful cells can be made without genetic engineering such as ex-vivo stimulated cytotoxic T lymphocytes. Thus, in certain embodiments, the target cell is not genetically modified.

### Separation Methods

The methods described herein utilize size based separation methods. Size based separation methods are those that are primarily based on the size or volume of a cell. Size based separation allows the separation of cells without affinity based reagents that may introduce sources of contamination or unwanted activation of the cells that are being separated. Size-based separation does not include those methods that rely primarily on an affinity based agent such as an antibody. Such affinity-based separations include the use of affinity agents coupled to beads (magnetic, paragenetic, or size based agarose or polystyrene, or polybrene beads). Affinity based methods generally rely on capture of one or more cells by a magnet or other affinity, covalent, or binding interaction (e.g., biotin-streptavidin). Size based selection methods can be used to separate the large cells, the small cells or both from the lymphocytes. Size based selection methods can be used to separate activated from inactivated lymphocytes. In certain embodiments, the size based selection methods used in the methods herein is any one or more of array-based Separation (including DLD), dielectrophoresis, acoustophoretic isolation, or density gradient separation. In certain embodiments, the size based selection methods used in the methods herein is any one or more of array-based Separation (including DLD), dielectrophoresis, or acoustophoretic isolation.

### Density Gradient Separation

Methods comprising centrifugal apheresis separates plasma from cellular components based on density can be useful for obtaining one or more cells from a sample. Density gradient separation apheresis devices are designed to separate plasma or blood components from whole blood, for the purposes of depletion or exchange of these components or plasma. Density gradient separation comprises drawing whole blood from a patient and separating the blood into its components, utilizing centrifugal force as the basis of operation. Centrifugal flow devices most commonly deliver continuous flow from the patient to the centrifuge. An anticoagulant, usually citrate, is added before centrifugation, which is then followed by return of the rest of the blood components with the appropriate replacement fluid (typically albumin or plasma) so that a continuous flow extracorporeal circuit is formed. Density gradient methods generally use a media with a higher density than water that includes neutral, highly branched, high-mass, hydrophilic polysaccharide. Such densities range from 1.0 to 1.2 g/ml. Such media are sold under a variety of names including Percoll™, Ficoll™, and Histopaque™.

### Array-Based Separation

Methods utilizing arrays comprising microstructures (e.g., microposts or columns) that construct pores that separate cells based on critical sizes. For example, such methods generally utilize size exclusion to prevent or restrict entrance or passage by physical blockage. Embodiments of size exclusion comprise the use of small pores to prevent large non-deformable particles from entering the pores. The pore size can be engineered to allow for the separation of particles of different sizes (critical sizes). Such methods can also utilize laminar flow, tangential flow, or cross flow dynamics to facilitate sample processing. Accordingly, density gradient separation can be used for the size based separation described herein, either to separate large cells, small cells, or activated from inactivated cells.

For example, methods comprising Deterministic Lateral Displacement (DLD) for separating different cell types can be useful for obtaining one or more target cells from a blood related sample. DLD is a process in which particles are deflected on a path through an array, deterministically, based on their size in relation to some of the array parameters. DLD can also be used to concentrate cells and for buffer exchange. Processes are generally described herein in terms of continuous flow (DC conditions, i.e., bulk fluid flow in only a single direction). However, DLD can also work under oscillatory flow (AC conditions, i.e., bulk fluid flow alternating between two directions). DLD generally functions to separate cells or components thereof base on the critical size or predetermined size of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced. When a profile of fluid flow through a gap is symmetrical about the plane that bisects the gap in the direction of bulk fluid flow, the critical size can be identical for both sides of the gap; however, when the profile is asymmetrical, the critical sizes of the two sides of the gap can differ.

Basic principles of size based microfluidic separations and the design of obstacle arrays for separating cells have been provided elsewhere (see, US 2014/0342375; US 2016/0139012; 7,318,902 and U.S. Pat. No. 7,150,812, which are hereby incorporated herein in their entirety) and are also summarized in the sections below.

Procedures for making and using microfluidic devices that are capable of separating cells on the basis of size have also been fully described in the art. Such devices include those described in U.S. Pat. Nos. 5,837,115; 7,150,812; 6,685,841; 7,318,902; 7,472,794; and 7,735,652; all of which are hereby incorporated by reference in their entirety. Other references that provide guidance that may be helpful in the making and use of devices for the present invention include: U.S. Pat. Nos. 5,427,663; 7,276,170; 6,913,697; 7,988,840; 8,021,614; 8,282,799; 8,304,230; 8,579,117; US 2006/0134599; US 2007/0160503; US 20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; US 2008/0113358; and WO2012094642 all of which are also incorporated by reference herein in their entirety.

Described herein are examples of a microfluidic device for separating target particles or target cells of a predetermined size from other constituents of a sample. The device may have a planar support that will typically be rectangular and can be made of any material compatible with a separation method, including silicon, glasses, hybrid materials or (preferably) polymers. The support may have a top surface and a bottom surface, one or both of which have at least one embedded channel extending from one or more sample inlets and one or more distinct fluid inlets, to one or more product outlets and one or more distinct waste outlets. Fluid inlets (as opposed to sample inlets) may sometimes be referred to as “buffer” or “wash” inlets and, depending on the objectives of a separation may be used to transport a variety of fluids into channels. Unless otherwise indicated by usage or context, it will be understood that a “fluid” may be a buffer, contain reagents, constitute growth medium for cells or generally be any liquid, and contain any components, compatible with operation of a device and the objectives of the user.

When fluid is applied to a device through a sample or fluid inlet, it flows through the channel toward the outlets, thereby defining a direction of bulk fluid flow. In order to separate cells or particles of different sizes, the channel includes an array of obstacles organized into columns that extend longitudinally along the channel (from inlet to outlet), and rows that extend laterally across the channel. Each subsequent row of obstacles is shifted laterally with respect to the previous row, thereby defining an array direction that deviates from the direction of bulk fluid flow by atilt angle (F). The obstacles are positioned so as to define a critical size such that when a sample is applied to an inlet of the device and flows to an outlet, particles or cells in the sample larger than the critical size follow in the array direction and particles smaller than the critical size flow the direction of bulk fluid flow, thereby resulting in a separation.

Adjacent obstacles in a row of the array are separated by a gap, G1, that is perpendicular to the direction of bulk fluid flow and adjacent obstacles in a column are separated by a gap, G2, which is parallel to the direction of bulk fluid flow. One characteristic of the present devices is that the ratio of the size of gap G2 to the size of gap G1 does not equal 1, with G1 typically being wider than G2 (e.g., by 10-100%). The obstacles in an array each have at least two vertices and are positioned so that each gap is flanked on either side by at least one vertex. In preferred embodiments, the vertices extend into parallel gaps so that the gaps are flanked on either side by one or more vertices pointing toward one another but not directly opposite one another and/or obstacles have vertices that extend into perpendicular gaps such that the gaps are flanked on either side by vertices pointing toward, and directly opposite to, one another.

In some embodiments, G1 and G2 may each independently be about 9 μm to about 30 μm. In some embodiments, G1 and G2 may each independently be about 9 μm to about 11 μm, about 9 μm to about 13 μm, about 9 μm to about 15 m, about 9 μm to about 17 μm, about 9 μm to about 19 μm, about 9 μm to about 21 μm, about 9 μm to about 22 μm, about 9 μm to about 24 μm, about 9 μm to about 26 μm, about 9 μm to about 28 μm, about 9 μm to about 30 μm, about 11 μm to about 13 μm, about 11 μm to about 15 μm, about 11 μm to about 17 μm, about 11 μm to about 19 μm, about 11 μm to about 21 μm, about 11 μm to about 22 μm, about 11 μm to about 24 μm, about 11 μm to about 26 μm, about 11 μm to about 28 μm, about 11 μm to about 30 μm, about 13 μm to about 15 μm, about 13 μm to about 17 μm, about 13 μm to about 19 μm, about 13 μm to about 21 μm, about 13 μm to about 22 μm, about 13 μm to about 24 μm, about 13 μm to about 26 μm, about 13 μm to about 28 μm, about 13 μm to about 30 μm, about 15 μm to about 17 μm, about 15 μm to about 19 μm, about 15 μm to about 21 μm, about 15 μm to about 22 μm, about 15 μm to about 24 μm, about 15 μm to about 26 μm, about 15 μm to about 28 μm, about 15 μm to about 30 μm, about 17 μm to about 19 μm, about 17 μm to about 21 μm, about 17 μm to about 22 μm, about 17 μm to about 24 μm, about 17 μm to about 26 μm, about 17 μm to about 28 μm, about 17 μm to about 30 μm, about 19 μm to about 21 μm, about 19 μm to about 22 μm, about 19 μm to about 24 μm, about 19 μm to about 26 μm, about 19 μm to about 28 μm, about 19 μm to about 30 μm, about 21 μm to about 22 μm, about 21 μm to about 24 μm, about 21 μm to about 26 μm, about 21 μm to about 28 μm, about 21 μm to about 30 μm, about 22 μm to about 24 μm, about 22 μm to about 26 μm, about 22 μm to about 28 μm, about 22 μm to about 30 μm, about 24 μm to about 26 μm, about 24 μm to about 28 μm, about 24 μm to about 30 μm, about 26 μm to about 28 μm, about 26 μm to about 30 μm, or about 28 μm to about 30 μm. In some embodiments, G1 and G2 may each independently be about 9 μm, about 11 μm, about 13 μm, about 15 μm, about 17 μm, about 19 μm, about 21 μm, about 22 μm, about 24 μm, about 26 μm, about 28 μm, or about 30 μm. In some embodiments, G1 and G2 may each independently be at least about 9 μm, about 11 μm, about 13 μm, about 15 μm, about 17 μm, about 19 μm, about 21 μm, about 22 μm, about 24 μm, about 26 μm, or about 28 μm. In some embodiments, G1 and G2 may each independently be at most about 11 μm, about 13 μm, about 15 μm, about 17 μm, about 19 μm, about 21 μm, about 22 μm, about 24 μm, about 26 μm, about 28 μm, about 29 μm, about 30 μm, about 31 μm, about 32 μm, about 33 μm, about 34 μm, or about 35 μm.

The microfluidic devices will also typically have an obstacle bonding layer that is bonded to a surface of the planar support and bonded to the obstacles in channels to prevent fluid or sample from flowing over obstacles during operation of the device. This obstacle bonding layer may comprise one or more passages fluidically connected to the inlets of the channel and to the outlets of the channel which permit the flow of fluid.

In general, the microfluidic devices will be used to separate target particles or target cells having a size larger than the critical size of the device from contaminants, fluids, non-target particles, or non-target cells with sizes smaller than the critical size. When a sample containing the target cells or particles is applied to a device through a sample inlet and fluidically passed through the channel, the target cells or target particles will flow to one or more product outlets where a product enriched in target cells or target particles is obtained. The term “enriched” as used in this context means that the ratio of target cells or particles to contaminants is higher in the product than in the sample. Contaminants, fluids, non-target particles, and non-target cells with a size smaller than the critical size will flow predominantly to one more waste outlets where they may be either collected or discarded.

Although the objective of a separation will generally be to separate target cells or particles from smaller contaminants, there may be times when a user wants to separate target cells or particles from larger contaminants. In these instances, a microfluidic device may be used with a critical size larger than the target cells or particles but smaller than the contaminants. Combinations of two or more obstacle arrays with different critical sizes, either on a single device or on multiple devices, may also be used in separations. For example, a device may have channels with a first array of obstacles that has a critical size larger than T cells but smaller than granulocytes and monocytes and a second array with a critical size smaller than T cells but larger than platelets and red blood cells. Processing of a blood sample on such a device allows for the collection of a product in which T cells have been separated from granulocytes, monocytes, platelets and red blood cells. The order of the obstacle arrays should not be of major importance to the result, i.e., an array with a smaller critical size could come before or after an array with a larger critical size. Also arrays with different critical sizes can be on separate devices that cells pass through.

Wide arrays and multiple outlets may be used for the collection multiple products, e.g., monocytes may be obtained at one outlet and T cells at a different outlet. Thus, using multiple arrays and multiple outlets may permit the concurrent collection of several products that are more purified than if a single array had been used. As further discussed below, high throughputs may be maintained by using many DLD arrays in parallel.

Preferably, the obstacles used in the microfluidic devices have a polygonal shape, with diamond or hexagonally shaped obstacles being preferred. The obstacles will also generally be elongated so that their length perpendicular to bulk fluid flow (P1) is different (generally longer) than their width parallel to bulk fluid flow (P2) by, for example, 10-100%. Typically, P1 will be longer than P2 by at least 15%, 30%, 50%, 100% or 150%. Expressed as a range, P1 may be 10-150% (15-100%; or 20-70%) longer than P2.

Microfluidic devices may also include a separator wall that extends from the sample inlet of a device, where it separates the sample inlet from fluid inlets and prevents mixing, into the array of obstacles in the channel. The separator wall is oriented parallel to the direction of bulk fluid flow and extends toward the sample and fluid outlets. The wall terminates before reaching the end of the channel, allowing sample and fluid streams to contact one another thereafter. It should generally extend at for a distance of at least 10% of the length of the array of obstacles but may extend for at least 20%, 40%, 60%, or 70% of the array. Expressed as a range the wall will typically extend for 10-70% of the length of the array of obstacles. More than one separator wall may also be present in a device and, depending on the objectives of a separation, may be positioned in different ways.

In order to increase the rate at which volume can be processed, a stacked separation assembly can be made by overlaying a first obstacle array with one or more stacked obstacle arrays, wherein the bottom surface of each stacked array is in contact with either the top surface, or an obstacle bonding layer on the top surface, of the first obstacle array or with the top surface, or the obstacle bonding layer on the top surface, of another array. Sample is provided to the sample inlets of all devices though a first common manifold and fluid is supplied to the fluid inlets through a second manifold that may or may not be the same as the first manifold. Product is removed from the product outlets through one or more product conduits and waste is removed from the waste outlets through one or more waste conduits that are different from the product conduits. In general, a stacked separation assembly will have 2 to 9 stacked arrays together with the first microfluidic obstacle array. However, a larger number of devices may also be used. In addition, the top surface of supports, and/or the bottom surface, may have multiple (e.g., 2-40 or 2-30) embedded channels and be used in purifying target particles or target cells.

Stacked separation assemblies may have a reservoir bonding layer which is attached to the bottom surface of the first microfluidic device and/or to the top surface of a stacked microfluidic device. The reservoir bonding layer should include a first end with one or more passages permitting the flow of fluid to inlets on the channels and optionally, one or more passages that permit the flow of fluid to, or from, the product and waste outlets of channels at a second end, opposite to first end and separated by material impermeable to fluid.

Stacked assemblies of devices may be supported in a cassette characterized by the presence of an outside casing with ports allowing for the transport of sample and fluids into the cassette and products and waste out of the cassette. The figure shows a cassette with two inlet ports and two outlet ports. However, multiple ports into and out of a cassette may be used and several products may be collected essentially simultaneously. It will also be recognized that cassettes can be part of a system in which there are components that are well known and commonly used in the art. Such common components include pumps, valves and processors for controlling fluid flow; sensors for monitoring system parameters such a flow rate and pressure; sensors for monitoring fluid characteristics such a pH or salinity; sensors for determining the concentration of cells or particles; and analyzers for determining the types of cells or particles present in the cassette or in material collected from the cassette. More generally, any equipment known in the art and compatible with the cassettes, the material being processed, and the processing objectives may be used.

### Designing Microfluidic Cartridges

The present disclosure provides microfluidic cartridges (i.e. devices, chips, cassettes, plates, microfluidic devices, cartridges, DLD devices, etc.) for purifying particles or cells. A microfluidic cartridge of the present disclosure may operate using a DLD method. A microfluidic cartridge of the present disclosure may be formed from a polymeric materials (e.g. thermoplastic), and may include one or more of a first planar support having a top surface and a bottom surface, and a second planar support having a top surface and a bottom surface, wherein the top surface of the first and second planar support comprises at least one embedded channel extending from one or more inlets to one or more outlets; the at least one embedded channel comprising an array of obstacles, wherein the bottom surface of the first and second planar support comprises a void space configured to be deformed when a the bottom of the first planar support is pressed to the bottom of the second planar support. A microfluidic cartridge of the present disclosure may be a single-use or disposable device. As an alternative, the microfluidic cartridge may be multi-use device. The use of polymers (e.g., thermoplastics) to form the microfluidic structure may allow for the use of an inexpensive and highly scalable soft embossing process while the void space may provide an improved ability to be manufactured quickly and avoid damage to the obstacles (i.e. posts, DLD arrays, etc.) during the manufacturing process.

The cartridges described herein may operate via deterministic lateral displacement, or DLD. DLD may include three different operating modes. The operating modes include: i) Separation, ii) Buffer Exchange and iii) Concentration. In each mode, particles above a critical diameter are deflected in the direction of the array from the point of entry, resulting in size selection, buffer exchange or concentration as a function of the geometry of the array. In all cases, particles below the critical diameter pass directly through the device under laminar flow conditions and subsequently off the device. The full length of the separation zone of the microfluidic cartridge may be about 75 mm and the width may be about 40 mm, with each individual channel being about 1.8 mm across.

The cartridges described herein may be arranged in a variety of orientations to accomplish different DLD modes or product outcomes. For example, four channels with side walls and an array of obstacles may be utilized. Samples containing blood, cells or particles may enter the channel through a sample inlet at the top and buffer, reagent or media may enter the channel at a separate fluid inlet. As they flow toward the bottom of the channels, cells or particles with sizes larger than the critical diameter of the array (>Dc) flow at angle that is determined by the array direction of the obstacles and are separated from cells and particles with sizes smaller than the critical diameter of the array (<Dc).

A cartridge may have a common sample inlet, e.g., for blood, which feeds the sample to inlets on each channel. There are separate inlets into channels for buffer, but which could, depending processing objectives, be used to introduce fluids with reagents, growth medium or other fluids into channels. At the bottom of each channel there is a product outlet which would typically be used for recovering target cells or particles that have sizes larger than the critical diameter of the obstacle arrays in the channels. The outlets from the individual channels feed into a common product outlet from which the target cells or particles can be recovered. Also shown are waste outlets in which cells and particles with sizes below the critical diameter of the obstacle arrays in the channels exit.

An embodiment of a cartridge may comprise 2 channels. The channels may have three sections designed to have progressively smaller diameter obstacles and gaps. Some cartridges may have a “bump array” having equilateral triangularly shaped obstacles disposed in a microfluidic channel. Equilateral triangular posts may be disposed in a parallelogram lattice arrangement that is tilted with respect to the directions of fluid flow. Other lattice arrangements (e.g., square, rectangular, trapezoidal, hexagonal, etc. lattices) can also be used. The tilt angle ϵ (epsilon) is chosen so the device is periodic. In some embodiments, a tilt angle of 18.4 degrees (⅓ radian) makes the device periodic after three rows. The tilt angle c also represents the angle by which the array direction is offset from the fluid flow direction. The gap between posts is denoted G with equilateral triangle side length S. Streamlines extend between the posts, dividing the fluid flow between the posts into three regions (“stream tubes”) of equal volumetric flow. A relatively large particle (having a size greater than the critical size for the array) follows the array tilt angle when fluid flow is in the direction shown. A relatively small particle (having a size smaller than the critical size for the array) follows the direction of fluid flow.

Cartridges describe herein may comprise a stacked separation assembly in which two microfluidic devices or cartridges are combined into a single unit. The topmost device may comprise a planar support that may be made using a variety of materials, but which is most preferably polymeric and which has a top surface and a bottom surface. The top surface of the support may contain reservoirs that provide sample inlets and inlets for buffer or other fluid at one end of the support and product outlets and waste outlets at the other end. Each reservoir may be fluidically connected through the support using small vias that connect the top surface to the channels on the bottom surface. The bottom surface of the support may have numerous embedded microfluidic channels each of which may have an array of obstacles connected by the channels. The embedded microfluidic layers may be bonded to an obstacle bonding layer that seals the first device and prevents fluid from flowing over the obstacles during operation. A second microfluidic device in the stack may contain embedded microfluidic channels on the topmost surface, and may be sealed by the same obstacle bonding layer as the topmost device. A reservoir bonding layer may have oblong openings allowing for the passage of liquid to channel inlets and the passage of liquid from channel outlets. The reservoir bonding layer may be similar to the obstacle bonding layer except that it attaches to a surface of a device and not obstacles and may be connected to one or more reservoirs feeding the stack of devices or to a manifold. Holes may be used for aligning the stacked devices. As described above, the two embedded microfluidic surfaces may face the same obstacle bonding layer. An alternate configuration would be to have the embedded channels on the top surface of both devices, with an intermediate layer between the devices that functions as both an obstacle bonding layer to the embedded channels below and a distribution layer to the reservoirs above. Multiple microfluidic devices may be stacked together to form a single assembly unit. At the top of this stack (and optionally both at the top and bottom) may be a manifold with feeds for a manifold inlet distributor and conduits leading from the manifold product outlet. Feeds leading to fluid inlets and conduits for removing fluid from waste outlets may also be present.

In certain examples, a device may have two channels where each channel has an array of asymmetrically spaced diamond obstacles, in which G1 is larger than G2. The diamonds may be offset so each successive row is shifted laterally relative to the previous row.

The present disclosure provides herein stacked assemblies of microfluidic devices inside a casing which together may be referred to as “cassettes” or a “cassette”. A port may serve as a feed for sample being fed through the casing and to a manifold. The port may be connected to manifold feeds which distribute sample through a manifold sample inlet to channel sample inlets. Once applied, sample flows through channels containing obstacle arrays and product having particles or cells larger than the critical size exit the stack of devices at a manifold product outlet. The product then flows from the manifold outlet through product conduits and is conveyed out of the cassette through product outlet port. Fluid flows into the cassette and to the manifold through port, which is connected to manifold fluid feeds. It may be distributed by a manifold fluid inlet to channel fluid inlets. The fluid flows through the channel and particles or cells smaller than the critical size exit the stack of devices predominantly through manifold waste outlet. These particles or cells then flow through waste conduits that convey waste out of the cassette through outlet port.

An embodiment of the cartridges or devices provided herein may comprise a channel bounded by two walls with a sample inlet and a fluid inlet. There may be a separator wall that prevents the sample flow stream from mixing with the fluid flow stream. The separator wall may extend into the obstacle array and end about halfway down. Initially after entering the obstacle array, the target cells may be diverted away from the direction of fluid flow until they reach the separator wall. They may then travel along the wall until it ends. Thereafter, they may resume being diverted until they exit the channel at the product outlet. Particles with sizes smaller than the critical size of the obstacle array are not diverted and exit the channel at the waste outlet. A channel may be bounded by walls with an inlet for sample, an inlet for a reagent and an inlet for buffer or other fluid. Sample may enter at the inlet and flow onto the obstacle array. There, particles or cells larger than the critical diameter of the array are diverted into the reagent stream where they undergo a reaction. A separator wall may run from the reagent inlet part way down the array of obstacles and may separate the reagent stream from the stream of buffer or other fluid. This wall maintains the cells or particles in the reagent stream for a longer period of time, thereby providing more time for reaction. At the end of the separator wall, the particles or cells resume being diverted to a product outlet where they may be collected. During this process the cells or particles are separated from unreacted reagent. A second separator wall may run from the end of the first separator wall to a waste outlet where buffer or other fluid, reagent and small particles or cells exit the device and may be collected or discarded. A second waste outlet may be used to remove reagent, fluid in which particles or cells in the sample were suspended and particles or cells smaller than the critical diameter of the obstacle array. These materials may be recovered or discarded.

G_{T} refers to the gap length between triangular posts, and G_{C} refers to the gap length between round posts. As the array tilt increases, the difference in gap lengths required for a particular critical size of the array (D_{C}), between triangular and circular posts, decreases.

The obstacle edge roundness (expressed as r/S) may have an effect on the critical size exhibited on the side of a gap bounded by the edge. Increasing roundness of a post increases the critical size value of that post fora given gap length.

In addition to critical size, posts of different shapes may also affect particle velocity given constant applied pressure. Given an applied pressure, arrays with triangular posts will result in a larger particle velocity than those with circular posts. Furthermore, the rate of particle velocity increase upon increasing pressure is also greater in triangular post arrays than circular post arrays.

Cartridges described herein may comprise a Seal/Lid on the top and/or bottom and a separation layer that comprises a plurality of obstacles that promote separation, a fluidic layer, and a void space or crumple zone that allows fabrication of the cartridge without deforming the plurality of obstacles. The plurality of obstacles may be arrayed in rows and columns, such that gaps configured to allow the passage of fluid and cells are formed. The obstacles may be arrayed such that they are stacked with no or minimal offset between repeating rows. Two or more cartridges may be stacked or connected in series or parallel to achieve greater separation or higher throughput.

As similar devices or microfluidic cartridges operate on a sub-millimeter scale and handles micro-liters, nano-liters, or smaller quantities of fluids, a major obstacle in manufacturing is avoiding damage or deformation of obstacles during embossing or assembly. For example, handling of the chip may result in pressure to the planar support, especially when planar supports are pressed together, which may then result in deformation or destruction of the planar support(s), obstacles (i.e. an array of obstacles), and the various separation lanes. Such deformation or destruction may result in a significant loss of performance in purifying particles or cells or may completely compromise the function of the microfluidic cartridge. In order to avoid potential deformations and defects during manufacturing and assembly, other microfluidic systems require slower manufacturing runs or accept diminished performance.

In an aspect, the present disclosure provides a microfluidic cartridge for purifying cells or particles. The microfluidic cartridge may include a first planar support. The first planar support may comprise a top surface and a bottom surface. The device may include a second planar support. The second planar support may comprise a top surface and a bottom surface. A top surface may comprise at least one embedded channel extending from one or more inlets to one or more outlets. The at least one embedded channel may comprise an array of obstacles. The bottom surface of the first and second planar support may comprise a void space. The void space may be configured to be deformed when the bottom of the first planar support is pressed to the bottom of the second planar support.

Separation according to this description occurs along a channel embedded in a planar support, the channel comprising a plurality of obstacles. For cartridges of this description a first and a second planar surface may be utilized. The first and second planar surfaces may be stacked (e.g., bottom to bottom or top to bottom with a spacer doubling the throughput and separation capacity while maintaining a small footprint. A top surface of a first and/or second planar surface may comprise at least 1 embedded channel to about 500 embedded channels. A top surface may comprise at least 1 embedded channel to about 2 embedded channels, 1 embedded channel to about 5 embedded channels, 1 embedded channel to about 20 embedded channels, 1 embedded channel to about 50 embedded channels, 1 embedded channel to about 100 embedded channels, 1 embedded channel to about 500 embedded channels, about 2 embedded channels to about 5 embedded channels, about 2 embedded channels to about 20 embedded channels, about 2 embedded channels to about 50 embedded channels, about 2 embedded channels to about 100 embedded channels, about 2 embedded channels to about 500 embedded channels, about 5 embedded channels to about 20 embedded channels, about 5 embedded channels to about 50 embedded channels, about 5 embedded channels to about 100 embedded channels, about 5 embedded channels to about 500 embedded channels, about 20 embedded channels to about 50 embedded channels, about 20 embedded channels to about 100 embedded channels, about 20 embedded channels to about 500 embedded channels, about 50 embedded channels to about 100 embedded channels, about 50 embedded channels to about 500 embedded channels, or about 100 embedded channels to about 500 embedded channels. A top surface may comprise at least 1 embedded channel, about 2 embedded channels, about 5 embedded channels, about 20 embedded channels, about 50 embedded channels, about 100 embedded channels, or about 500 embedded channels. A top surface may comprise at least 1 embedded channel, about 2 embedded channels, about 5 embedded channels, about 20 embedded channels, about 50 embedded channels, or about 100 embedded channels. A top surface may comprise at least at most about 2 embedded channels, about 5 embedded channels, about 20 embedded channels, about 50 embedded channels, about 100 embedded channels, or about 500 embedded channels. Atop surface or a first or second planar surface may comprise about 28 channels (56 when stacked). An additional third, fourth, fifth, or sixth planar surface may also comprise a similar amount of embedded channels as the first or second planar surface.

The microfluidic cartridge may comprise at least 1 inlet to about 50 inlets. The microfluidic cartridge may comprise at least 1 inlet to about 2 inlets, 1 inlet to about 5 inlets, 1 inlet to about 10 inlets, 1 inlet to about 20 inlets, 1 inlet to about 50 inlets, about 2 inlets to about 5 inlets, about 2 inlets to about 10 inlets, about 2 inlets to about 20 inlets, about 2 inlets to about 50 inlets, about 5 inlets to about 10 inlets, about 5 inlets to about 20 inlets, about 5 inlets to about 50 inlets, about 10 inlets to about 20 inlets, about 10 inlets to about 50 inlets, or about 20 inlets to about 50 inlets. The microfluidic cartridge may comprise at least 1 inlet, about 2 inlets, about 5 inlets, about 10 inlets, about 20 inlets, or about 50 inlets. The microfluidic cartridge may comprise at least 1 inlet, about 2 inlets, about 5 inlets, about 10 inlets, or about 20 inlets. The microfluidic cartridge may comprise at least at most about 2 inlets, about 5 inlets, about 10 inlets, about 20 inlets, or about 50 inlets. The inlets may be fed by a common fluidic system or a dual fluidic system (one for buffer/diluent and one for sample).

The microfluidic cartridge may comprise at least 1 outlet to about 50 outlets. The microfluidic cartridge may comprise at least 1 outlet to about 2 outlets, 1 outlet to about 5 outlets, 1 outlet to about 10 outlets, 1 outlet to about 20 outlets, 1 outlet to about 50 outlets, about 2 outlets to about 5 outlets, about 2 outlets to about 10 outlets, about 2 outlets to about 20 outlets, about 2 outlets to about 50 outlets, about 5 outlets to about 10 outlets, about 5 outlets to about 20 outlets, about 5 outlets to about 50 outlets, about 10 outlets to about 20 outlets, about 10 outlets to about 50 outlets, or about 20 outlets to about 50 outlets. The microfluidic cartridge may comprise at least 1 outlet, about 2 outlets, about 5 outlets, about 10 outlets, about 20 outlets, or about 50 outlets. The microfluidic cartridge may comprise at least 1 outlet, about 2 outlets, about 5 outlets, about 10 outlets, or about 20 outlets. The microfluidic cartridge may comprise at least at most about 2 outlets, about 5 outlets, about 10 outlets, about 20 outlets, or about 50 outlets. The outlets may feed a common fluidic system or a dual fluidic system (one for waste and one for enriched target cells or particles).

The cartridge comprising two or more planar surfaces may comprise a void space to protect the array of obstacles in the lanes as their small size leads their susceptibility to deformation, leading to malfunction.

The void space of the microfluidic cartridge may be configured to deform, bend, swell, collapse, or crumple. The void space may be configured to protect the obstacles, channels, inlets, outlets, planar surfaces, or any combination thereof, from damage, displacement, deformation, or malfunction. The void space may comprise a crumple zone that is configured to protect the obstacles, channels, inlets, outlets, planar surfaces, or any combination thereof, from damage, displacement, deformation, or malfunction. The void space may have a volume of about 1 cubic μm to about 10,000 cubic μm. The void space may have a volume of about 1 cubic μm to about 5 cubic μm, about 1 cubic μm to about 10 cubic μm, about 1 cubic μm to about 30 cubic μm, about 1 cubic μm to about 50 cubic μm, about 1 cubic μm to about 100 cubic μm, about 1 cubic μm to about 300 cubic μm, about 1 cubic μm to about 1,000 cubic μm, about 1 cubic μm to about 3,000 cubic μm, about 1 cubic μm to about 10,000 cubic μm, about 5 cubic μm to about 10 cubic μm, about 5 cubic μm to about 30 cubic μm, about 5 cubic μm to about 50 cubic μm, about 5 cubic μm to about 100 cubic μm, about 5 cubic μm to about 300 cubic μm, about 5 cubic μm to about 1,000 cubic μm, about 5 cubic μm to about 3,000 cubic μm, about 5 cubic μm to about 10,000 cubic μm, about 10 cubic μm to about 30 cubic μm, about 10 cubic μm to about 50 cubic μm, about 10 cubic μm to about 100 cubic μm, about 10 cubic μm to about 300 cubic μm, about 10 cubic μm to about 1,000 cubic μm, about 10 cubic μm to about 3,000 cubic μm, about 10 cubic μm to about 10,000 cubic μm, about 30 cubic μm to about 50 cubic μm, about 30 cubic μm to about 100 cubic μm, about 30 cubic μm to about 300 cubic μm, about 30 cubic μm to about 1,000 cubic μm, about 30 cubic μm to about 3,000 cubic μm, about 30 cubic μm to about 10,000 cubic μm, about 50 cubic μm to about 100 cubic μm, about 50 cubic μm to about 300 cubic μm, about 50 cubic μm to about 1,000 cubic μm, about 50 cubic μm to about 3,000 cubic μm, about 50 cubic μm to about 10,000 cubic μm, about 100 cubic μm to about 300 cubic μm, about 100 cubic μm to about 1,000 cubic μm, about 100 cubic μm to about 3,000 cubic μm, about 100 cubic μm to about 10,000 cubic μm, about 300 cubic μm to about 1,000 cubic μm, about 300 cubic μm to about 3,000 cubic μm, about 300 cubic μm to about 10,000 cubic μm, about 1,000 cubic μm to about 3,000 cubic μm, about 1,000 cubic μm to about 10,000 cubic μm, or about 3,000 cubic μm to about 10,000 cubic μm. The void space may have a volume of about 1 cubic μm, about 5 cubic μm, about 10 cubic μm, about 30 cubic μm, about 50 cubic μm, about 100 cubic μm, about 300 cubic μm, about 1,000 cubic μm, about 3,000 cubic μm, or about 10,000 cubic μm. The void space may have a volume of at least about 1 cubic μm, about 5 cubic μm, about 10 cubic μm, about 30 cubic μm, about 50 cubic μm, about 100 cubic μm, about 300 cubic μm, about 1,000 cubic μm, or about 3,000 cubic μm. The void space may have a volume of at most about 5 cubic μm, about 10 cubic μm, about 30 cubic μm, about 50 cubic μm, about 100 cubic μm, about 300 cubic μm, about 1,000 cubic μm, about 3,000 cubic μm, or about 10,000 cubic μm.

The bottom surface of a cartridge may comprise a plurality of void spaces shown here arranged into strips that run parallel with the length of the planar support. The void spaces may run beneath the array or column of obstacles or the lanes formed by the columns of obstacles fabricated on the top surface of the planar support. The top surface of the planar support may comprise a plurality of individual obstacles formed into arrays or columns creating gaps to allow the flow of fluid, cells, and/or particles. Beneath the obstacles embedded in the bottom surface of the planar support may be a void space. The area of the void space (length×width) opposite the lane can be at least about 80% of the area (length×width) of the lane. In certain embodiments, the area of the void space (length×width) opposite the lane can be at least about 90%, 100%, 110%, or 120% up to and including about 150% of the area (length×width) of the lane.

In one configuration the void spaces of the two planar supports may be symmetrical or nearly symmetrical and pressed back to back. However alternative arrangements are also possible, such as stacked with the void space above or below the obstacle layer.

The void space may be separated into two or more void spaces. The void space may be separated into at least 3, 4, 5, 6, 7, 8, 9, 10, 11, 12, 13, 14, 15, 16, 17, 18, 19, 20, 21, 22, 23, 24, 25, 26, 27, 28, 29, or 30 void spaces. The void space may be separated into exactly two void spaces. There may be a 1:1 ratio between channels or lanes and void spaces for each planar support comprising obstacles.

The planar support may be fabricated from two layers of material bonded together. The layers may be bonded together by adhesive, polymer, or thermoplastic. The layers may be comprised of polymer or thermoplastic. The polymer or thermoplastic layers or bonding material may be comprised of high-density polyethylene (HDPE), polypropylene (PP), polyethylene terephthalate (PT), polycarbonate (PC), or cyclic olefin copolymer (COC).

The top layer of a cartridge may comprise an array of obstacles in at least one embedded channel, void space, at least one inlet, at least one outlet, or combination thereof. The bottom layer of a cartridge may comprise an array of obstacles in at least one embedded channel, void space, at least one inlet, at least one outlet, or combination thereof. The layers may be positioned to where the planar supports are bonded together on their side surfaces, bottom surfaces, or top surfaces. The void space may be inside the interface of the planar supports bonded together, or outside the interface.

The microfluidic cartridge may further comprise an obstacle bonding layer that is bonded to the surface of the planar support and a top surface of the array of obstacles in the embedded channels to prevent fluid or sample from flowing over the array of obstacles during operation of the cartridge. The obstacle bonding layer may be metallic, polymer, or thermoplastic. The obstacle bonding layer may be a cover or a film. The polymer or thermoplastic layers or bonding material may be comprised of high-density polyethylene (HDPE), polypropylene (PP), polyethylene terephthalate (PT), polycarbonate (PC), or cyclic olefin copolymer (COC). The microfluidic cartridge may comprise two obstacle bonding layers on the outside of the top planar support. The microfluidic cartridge may comprise a single obstacle bonding layer in the middle of the cartridge as the bonding agent for the planar supports. The obstacle bonding layer may comprise one or more passages fluidically connected to the one or more inlets of the embedded channels which permit the flow of sample into the channels and one or more passages fluidically connected to the one or more outlets of the channels that permit the flow of fluid out from the one or more outlets. Such an obstacle layer may comprise at least about 1, at least about 2, at least about 3, at least about 4, at least about 5, at least about 10, at least about 20, at least about 30, at least about 50, or at least about 100 passages fluidically connected to the one or more inlets or one or more outlets of the embedded channels.

The microfluidic cartridge may have the obstacles positioned so as to define a critical size of the cartridge such that when a sample is applied to an inlet of the cartridge and flows to an outlet, particles or cells in the sample larger than the critical size are separated from particles or cells in the sample smaller than the critical size. Each obstacle may have its own individual sub-critical size, the sum the individual obstacles defining the critical size of the cartridge. The one or more outlets of the cartridge may comprise at least one product outlet, wherein target particles or cells, having a size larger than the critical size of the cartridge, are directed to the at least one product outlet. The one or more outlets of the cartridge may comprise at least one product outlet, wherein target particles or cells, having a size larger than the critical size of the cartridge, are directed to the at least one product outlet. The cartridge may have at least about 1, at least about 2, at least about 3, at least about 5, at least about 10, or at least about 50 product outlets. The particles, or cells, having a size larger than the critical size, may flow to the at least one product outlet. The cartridge may have at least about 1, at least about 2, at least about 3, at least about 5, at least about 10, or at least about 50 waste outlets.

The obstacles used in the cartridge may take the shape of columns or be triangular, square, rectangular, diamond shaped, trapezoidal, hexagonal, teardrop shaped, circular shape, semicircular shape, triangular with top side horizontal shape, and triangular with bottom side horizontal shape. In addition, adjacent obstacles may have a geometry such that the portions of the obstacles defining the gap are either symmetrical or asymmetrical about the axis of the gap that extends in the direction of bulk fluid flow. The obstacles may have vertices that extend into parallel gaps such that the gaps are flanked on either side by one or more vertices pointing toward one another but not directly opposite one another. The obstacles may have vertices that extend into perpendicular gaps such that the gaps are flanked on either side by vertices pointing toward one another and that are directly opposite one another. Obstacle location and shape can vary in a single chip. Additional obstacles can be added to any location of the device for any specific requirement. Also, the shape of the obstacle can be different in a device. Any combinations of posts shape, size and location can be used for specific requirement. The cartridge may be comprised of only diamond or hexagonal shaped obstacles.

The obstacle shapes may be elongated perpendicularly to the direction of fluid flow such that they have a horizontal length (P1) that is different from their vertical length (P2). P1 may have a length of about 1 μm to about 160 μm. P1 may have a length of about 1 μm to about 10 μm, about 1 μm to about 15 μm, about 1 μm to about 30 μm, about 1 μm to about 40 μm, about 1 μm to about 80 μm, about 1 μm to about 160 μm, about 10 μm to about 15 μm, about 10 μm to about 30 μm, about 10 μm to about 40 μm, about 10 μm to about 80 μm, about 10 μm to about 160 μm, about 15 μm to about 30 μm, about 15 μm to about 40 μm, about 15 μm to about 80 μm, about 15 μm to about 160 μm, about 30 μm to about 40 μm, about 30 μm to about 80 μm, about 30 μm to about 160 μm, about 40 μm to about 80 μm, about 40 μm to about 160 μm, or about 80 μm to about 160 μm. P1 may have a length of about 1 μm, about 10 μm, about 15 μm, about 30 μm, about 40 μm, about 80 μm, or about 160 μm. P1 may have a length of at least about 1 μm, about 10 μm, about 15 μm, about 30 μm, about 40 μm, or about 80 μm. P1 may have a length of at most about 10 μm, about 15 μm, about 30 μm, about 40 μm, about 80 μm, or about 160 μm. P2 may have a length of about 1 μm to about 160 μm. P2 may have a length of about 1 μm to about 10 μm, about 1 μm to about 15 μm, about 1 μm to about 30 μm, about 1 μm to about 40 μm, about 1 μm to about 80 μm, about 1 μm to about 160 μm, about 10 μm to about 15 μm, about 10 μm to about 30 μm, about 10 μm to about 40 μm, about 10 μm to about 80 μm, about 10 μm to about 160 μm, about 15 μm to about 30 μm, about 15 μm to about 40 μm, about 15 μm to about 80 μm, about 15 μm to about 160 μm, about 30 μm to about 40 μm, about 30 μm to about 80 μm, about 30 μm to about 160 μm, about 40 μm to about 80 μm, about 40 μm to about 160 μm, or about 80 μm to about 160 μm. P2 may have a length of about 1 μm, about 10 μm, about 15 μm, about 30 μm, about 40 μm, about 80 μm, or about 160 μm. P2 may have a length of at least about 1 μm, about 10 μm, about 15 μm, about 30 μm, about 40 μm, or about 80 μm. P2 may have a length of at most about 10 μm, about 15 μm, about 30 μm, about 40 μm, about 80 μm, or about 160 μm. P1 may be longer than P2 by about 25% to about 200%. P1 may be longer than P2 by about 25% to about 50%, about 25% to about 75%, about 25% to about 100%, about 25% to about 150%, about 25% to about 200%, about 50% to about 75%, about 50% to about 100%, about 50% to about 150%, about 50% to about 200%, about 75% to about 100%, about 75% to about 150%, about 75% to about 200%, about 100% to about 150%, about 100% to about 200%, or about 150% to about 200%. P1 may be longer than P2 by about 25%, about 50%, about 75%, about 1000%, about 150%, or about 200%. P1 may be longer than P2 by at least about 25%, about 50%, about 75%, about 100%, or about 150%. P1 may be longer than P2 by at most about 50%, about 75%, about 100%, about 150%, or about 200%.

The microfluidic cartridge may comprise obstacles as an array of obstacles. The obstacles may be arranged in in columns and in rows that form discreet arrays. The array of obstacles may compromise at least about 5 columns to about 50 columns. The array of obstacles may compromise at least about 5 columns to about 10 columns, about 5 columns to about 28 columns, about 5 columns to about 29 columns, about 5 columns to about 30 columns, about 5 columns to about 50 columns, about 10 columns to about 28 columns, about 10 columns to about 29 columns, about 10 columns to about 30 columns, about 10 columns to about 50 columns, about 28 columns to about 29 columns, about 28 columns to about 30 columns, about 28 columns to about 50 columns, about 29 columns to about 30 columns, about 29 columns to about 50 columns, or about 30 columns to about 50 columns. The array of obstacles may compromise at least about 5 columns, about 10 columns, about 28 columns, about 29 columns, about 30 columns, or about 50 columns. The array of obstacles may compromise at least about 5 columns, about 10 columns, about 28 columns, about 29 columns, or about 30 columns. The array of obstacles may compromise at least at most about 10 columns, about 28 columns, about 29 columns, about 30 columns, or about 50 columns. The array of obstacles may compromise at least about 20 rows to about 500 rows. The array of obstacles may compromise at least about 20 rows to about 30 rows, about 20 rows to about 60 rows, about 20 rows to about 100 rows, about 20 rows to about 200 rows, about 20 rows to about 500 rows, about 30 rows to about 60 rows, about 30 rows to about 100 rows, about 30 rows to about 200 rows, about 30 rows to about 500 rows, about 60 rows to about 100 rows, about 60 rows to about 200 rows, about 60 rows to about 500 rows, about 100 rows to about 200 rows, about 100 rows to about 500 rows, or about 200 rows to about 500 rows. The array of obstacles may compromise at least about 20 rows, about 30 rows, about 60 rows, about 100 rows, about 200 rows, or about 500 rows. The array of obstacles may compromise at least about 20 rows, about 30 rows, about 60 rows, about 100 rows, or about 200 rows. The array of obstacles may compromise at least at most about 30 rows, about 60 rows, about 100 rows, about 200 rows, or about 500 rows. Multiple arrays of obstacles can be arranged in discrete lanes. The array of obstacles of the first or second planar support forms about 10 lanes to about 50 lanes. The array of obstacles of the first or second planar support forms about 10 lanes to about 20 lanes, about 10 lanes to about 28 lanes, about 10 lanes to about 30 lanes, about 10 lanes to about 50 lanes, about 20 lanes to about 28 lanes, about 20 lanes to about 30 lanes, about 20 lanes to about 50 lanes, about 28 lanes to about 30 lanes, about 28 lanes to about 50 lanes, or about 30 lanes to about 50 lanes. The array of obstacles of the first or second planar support forms about 10 lanes, about 20 lanes, about 28 lanes, about 30 lanes, or about 50 lanes. The array of obstacles of the first or second planar support forms at least about 10 lanes, about 20 lanes, about 28 lanes, or about 30 lanes. The array of obstacles of the first or second planar support forms at most about 20 lanes, about 28 lanes, about 30 lanes, or about 50 lanes.

Each cartridge may comprise at least one, at least two, at least three, or at least four sets of arrays of obstacles. Each planar top surface may comprise at least one or at least two arrays. The cartridge may comprise a total of about 20 lanes to about 100 lanes. The cartridge may comprise a total of about 20 lanes to about 40 lanes, about 20 lanes to about 56 lanes, about 20 lanes to about 60 lanes, about 20 lanes to about 100 lanes, about 40 lanes to about 56 lanes, about 40 lanes to about 60 lanes, about 40 lanes to about 100 lanes, about 56 lanes to about 60 lanes, about 56 lanes to about 100 lanes, or about 60 lanes to about 100 lanes. The cartridge may comprise a total of about 20 lanes, about 40 lanes, about 56 lanes, about 60 lanes, or about 100 lanes. The cartridge may comprise a total of at least about 20 lanes, about 40 lanes, about 56 lanes, or about 60 lanes. The cartridge may comprise a total of at most about 40 lanes, about 56 lanes, about 60 lanes, or about 100 lanes.

The inlets, outlets, or both, of the microfluidic cartridge may be in fluid connection with pumps or motors to drive the flow of fluids within and outside of the cartridge. The inlets, outlets, or both, may be fluidically connected to at least about 1, 2, 3, 4, 5, 6, 7, 8, 9, or 10 pumps. The pumps may be peristaltic pumps. The pumps may be fluidically connected to each other or isolated. The inlets and outlets of the cartridge may be in fluidic connection with two peristaltic pumps connected in parallel to each other. The inlets and outlets of the cartridge may be in fluidic connection with two peristaltic pumps connected in serial to each other.

The microfluidic cartridge may be fabricated from a metal, polymer, or thermoplastic. The polymer or thermoplastic may be comprised of high-density polyethylene (HDPE), polypropylene (PP), polyethylene terephthalate (PT), polycarbonate (PC), or cyclic olefin copolymer (COC). In an example, the microfluidic cartridge is comprised of cyclic olefin copolymer.

The present disclosure also provides for a microfluid assembly comprising a plurality of microfluidic cartridges in fluidic connection. The cartridges in the assembly may be stacked or layered. The plurality of microfluidic cartridges may comprise at least about 2, 3, 4, 5, 6, 7, 8, 9, 10, 20, or 30 cartridges. The plurality of cartridges may be fluidically connected in serial or in parallel.

During DLD, a fluid sample containing cells is introduced into a device at an inlet and is carried along with fluid flowing through the device to outlets. As cells in the sample traverse the device, they encounter posts or other obstacles that have been positioned to form gaps or pores through which the cells must pass. Each successive row of obstacles is displaced relative to the preceding row so as to form an array direction that differs from the direction of fluid flow in the flow channel. The “tilt angle” defined by these two directions, together with the width of gaps between obstacles, the shape of obstacles, and the orientation of obstacles forming gaps are primary factors in determining a “critical size” for an array. Cells having a size greater than the critical size travel in the array direction, rather than in the direction of bulk fluid flow and particles having a size less than the critical size travel in the direction of bulk fluid flow. In devices used for leukapheresis-derived compositions, array characteristics may be chosen that result in white blood cells being diverted in the array direction whereas red blood cells and platelets continue in the direction of bulk fluid flow. In order to separate a chosen type of leukocyte from others having a similar size, a carrier may then be used that binds to that cell in a way that promotes DLD separation and which thereby results in a complex that is larger than uncomplexed leukocytes. It may then be possible to carry out a separation on a device having a critical size smaller than the complexes but bigger than the uncomplexed cells.

A device can be made using any of the materials from which micro- and nano-scale fluid handling devices are typically fabricated, including silicon, glasses, plastics, and hybrid materials. A diverse range of thermoplastic materials suitable for microfluidic fabrication is available, offering a wide selection of mechanical and chemical properties that can be leveraged and further tailored for specific applications. In an aspect, the microfluidic cartridge may be fabricated by soft embossing and UV-light curing.

The microfluidic cartridge (or device, cassette, chip, etc.) may be made by techniques including Replica molding, Soft lithography with PDMS, Thermoset polyester, Embossing, soft embossing, hot embossing, Roll to Roll embossing, Injection Molding, Laser Ablation, UV-light curing, and combinations thereof. Further details can be found in “Disposable microfluidic devices: fabrication, function and application” by Fiorini, et al. (BioTechniques 38:429-446 (March 2005)), which is hereby incorporated by reference herein in its entirety. The book “Lab on a Chip Technology” edited by Keith E. Herold and Avraham Rasooly, Caister Academic Press Norfolk UK (2009) is another resource for methods of fabrication and is hereby incorporated by reference herein in its entirety.

High-throughput embossing methods such as reel-to-reel processing of thermoplastics is an attractive method for industrial microfluidic chip production. The use of single chip hot embossing can be a cost-effective technique for realizing high-quality microfluidic devices during the prototyping stage. Methods for the replication of microscale features in two thermoplastics, polymethylmethacrylate (PMMA) and/or polycarbonate (PC), are described in “Microfluidic device fabrication by thermoplastic hot-embossing” by Yang, et al. (Methods Mol. Biol. 949: 115-23 (2013)), which is hereby incorporated by reference herein in its entirety

The flow channel can be constructed using two or more pieces which, when assembled, form a closed cavity (preferably one having orifices for adding or withdrawing fluids) having the obstacles disposed within it. The obstacles can be fabricated on one or more pieces that are assembled to form the flow channel, or they can be fabricated in the form of an insert that is sandwiched between two or more pieces that define the boundaries of the flow channel.

The obstacles may be solid bodies that extend in an array laterally across the flow channel and longitudinally along the channel from the inlets to the outlets. Where an obstacle is integral with (or an extension of) one of the faces of the flow channel at one end of the obstacle, the other end of the obstacle can be sealed to or pressed against the opposite face of the flow channel. A small space (preferably too small to accommodate any particles of interest for an intended use) is tolerable between one end of an obstacle and a face of the flow channel, provided the space does not adversely affect the structural stability of the obstacle or the relevant flow properties of the device.

Surfaces can be coated to modify their properties and polymeric materials employed to fabricate devices, can be modified in many ways. In some cases, functional groups such as amines or carboxylic acids that are either in the native polymer or added by means of wet chemistry or plasma treatment are used to crosslink proteins or other molecules. DNA can be attached to COC and PMMA substrates using surface amine groups. Surfactants such as Pluronic® can be used to make surfaces hydrophilic and protein repellant by adding Pluronic® to PDMS formulations. In some cases, a layer of PMMA is spin coated on a device, e.g., microfluidic chip and PMMA is “doped” with hydroxypropyl cellulose to vary its contact angle.

To reduce non-specific adsorption of cells or compounds, e.g., released by lysed cells or found in biological samples, onto the channel walls, one or more walls may be chemically modified to be non-adherent or repulsive. The walls may be coated with a thin film coating (e.g., a monolayer) of commercial non-stick reagents, such as those used to form hydrogels. Additional examples of chemical species that may be used to modify the channel walls include oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers may also be employed to repel oppositely charged species. The type of chemical species used for repulsion and the method of attachment to the channel walls can depend on the nature of the species being repelled and the nature of the walls and the species being attached. Such surface modification techniques are well known in the art. The walls may be functionalized before or after the device is assembled.

IV. Separation Processes that Use DLD

The DLD devices described herein can be used to purify cells, cellular fragments, cell adducts, or nucleic acids. Separation and purification of blood components using devices can be found, for example, in US Publication No. US 2016/0139012, the teaching of which is incorporated by reference herein in its entirety.

The purity, yields and viability of cells produced by DLD methods will vary based on a number of factors including the nature of the starting material, the exact procedure employed and the characteristics of the DLD device. Preferably, purifications, yields and viabilities of at least 60% should be obtained with, higher percentages, at least 70, 80 or 90% being more preferred.

In an aspect, the present disclosure provides methods for enriching target particles or target cells of a predetermined size from contaminants in a sample. Methods for enriching target particles or target cells use any cartridge, microfluidic cartridge, cassette, chip, device, fluidic device, or microfluidic device as described elsewhere herein. A method may comprise obtaining a sample comprising target particles or target cells and the contaminants. The method may further comprise separating the target particles or target cells from the contaminants by applying the sample to one or more sample inlets on any of the cartridges, cassettes, or devices described herein. The method may further comprise flowing the sample to the outlets on any of the cartridges, cassettes, or devices described herein. The method may further comprise obtaining a product enriched in target particles or target cells from one or more outlets while removing the contaminants. The method may result in a superior ability to purify or separate cells or particles from contaminants, creating greater cells yields, improved ability to expand the product in vitro, and an enriched cell product more amenable to transduction or other genetic engineering.

The method may entail the used of deterministic lateral displacement whereby the device has a critical size as described herein and the contaminants and the target particles or target cells are separated on the basis of having different critical size. The method may comprise flowing a sample containing the target particles or target cells and contaminants to any of the of the cartridges, cassettes, or devices described herein, wherein the target particles or target cells have a size larger than a critical size of the array of obstacles and at least some contaminants have sizes smaller than the critical size of the array of obstacles and wherein target cells or target particles flow to the one or more product outlets where a product enriched in target cells or target particles is obtained and contaminants with a size smaller than the critical size of the array of obstacles flow to one more waste outlets. The method may comprise flowing a sample containing the target particles or target cells and contaminants to any of the of the cartridges, cassettes, or devices described herein, wherein the target particles or target cells have a size smaller than a critical size of the array of obstacles and at least some contaminants have sizes larger than the critical size of the array of obstacles and wherein target cells or target particles flow to the one or more product outlets where a product enriched in target cells or target particles is obtained and contaminants with a size larger than the critical size of the array of obstacles flow to one more waste outlets.

The method may comprise flowing a sample containing the target particles or target cells and contaminants to any of the of the cartridges, cassettes, or devices described herein, at a constant flow rate or a variable flowrate. The cartridge flow rate of the method may be about 400 mL per hour. The cartridge flow rate of the method may be about 100 mL per hour to about 1,000 mL per hour. The cartridge flow rate of the method may be about 100 mL per hour to about 200 mL per hour, about 100 mL per hour to about 400 mL per hour, about 100 mL per hour to about 800 mL per hour, about 100 mL per hour to about 1,000 mL per hour, about 200 mL per hour to about 400 mL per hour, about 200 mL per hour to about 800 mL per hour, about 200 mL per hour to about 1,000 mL per hour, about 400 mL per hour to about 800 mL per hour, about 400 mL per hour to about 1,000 mL per hour, or about 800 mL per hour to about 1,000 mL per hour. The cartridge flow rate of the method may be about 100 mL per hour, about 200 mL per hour, about 400 mL per hour, about 800 mL per hour, or about 1,000 mL per hour. The cartridge flow rate of the method may be at least about 100 mL per hour, about 200 mL per hour, about 400 mL per hour, or about 800 mL per hour. The cartridge flow rate of the method may be at most about 200 mL per hour, about 400 mL per hour, about 800 mL per hour, or about 1,000 mL per hour.

The method may comprise an internal pressure within the cartridge. The internal pressure of the cartridge may be at least about 15 pounds per square inch. The internal pressure of the cartridge may be at least about 1.5 pounds per square inch to about 50 pounds per square inch. The internal pressure of the cartridge may be at least about 1.5 pounds per square inch to about 5 pounds per square inch, about 1.5 pounds per square inch to about 10 pounds per square inch, about 1.5 pounds per square inch to about 15 pounds per square inch, about 1.5 pounds per square inch to about 20 pounds per square inch, about 1.5 pounds per square inch to about 50 pounds per square inch, about 5 pounds per square inch to about 10 pounds per square inch, about 5 pounds per square inch to about 15 pounds per square inch, about 5 pounds per square inch to about 20 pounds per square inch, about 5 pounds per square inch to about 50 pounds per square inch, about 10 pounds per square inch to about 15 pounds per square inch, about 10 pounds per square inch to about 20 pounds per square inch, about 10 pounds per square inch to about 50 pounds per square inch, about 15 pounds per square inch to about 20 pounds per square inch, about 15 pounds per square inch to about 50 pounds per square inch, or about 20 pounds per square inch to about 50 pounds per square inch. The internal pressure of the cartridge may be at least about 1.5 pounds per square inch, about 5 pounds per square inch, about 10 pounds per square inch, about 15 pounds per square inch, about 20 pounds per square inch, or about 50 pounds per square inch. The internal pressure of the cartridge may be at least about 1.5 pounds per square inch, about 5 pounds per square inch, about 10 pounds per square inch, about 15 pounds per square inch, or about 20 pounds per square inch. The internal pressure of the cartridge may be at least at most about 5 pounds per square inch, about 10 pounds per square inch, about 15 pounds per square inch, about 20 pounds per square inch, or about 50 pounds per square inch.

Separation of cells in a sample can be performed by positive or negative selection of cell types using DLD and be collected in an output tube. Accordingly, DLD can be used for the size-based separation described herein, either to separate large cells, small cells, or activated from inactivated cells.

### Dielectrophoresis

Methods comprising dielectrophoresis (DEP) for separating different cell types can be useful for obtaining one or more target cells from a blood related sample. Dielectrophoresis (DEP) is a phenomenon in which particles, or cells, exposed to the gradient of an electric field are polarized depending on the characteristics of the cells and the medium that surrounds them. See U.S. Pat. No. 10,078,066; See also Douglas T A et al. “Separation of Macrophages and Fibroblasts Using Contactless Dielectrophoresis and a Novel ImageJ Macro.” Bioelectricity. 2019; 1(1):49-55. doi:10.1089/bioe.2018.0004. Such polarization induces movement of the cells along the gradient of the electric field. Accordingly, dielectrophoresis (DEP) can be used to trap cells or divert them from normal streamlines. For example, dielectrophoresis (DEP) can be used to positively or negatively select target cell from a population of cells. Contactless dielectrophoresis (DEP), which employs a polydimethylsiloxane (PDMS) microfluidic device containing a cell flow chamber can be used to facilitate dielectrophoresis (DEP) isolation of cell types. A polydimethylsiloxane (PDMS) microfluidic device generally comprises a chamber containing an array of 20 micrometer (um) posts where cells trap based on the gradient of an applied electric field. The device also generally comprises contactless fluidic electrodes that are filled with conductive fluid and separated from the main channel by a thin poly dimethylsiloxane (PDMS) membrane. Applying voltage using contactless electrodes filled with a concentrated buffer (e.g. 10× concentrated phosphate-buffered saline (PBS)) eliminates problems with cell mortality as is seen in traditional dielectrophoresis by preventing electrolysis and bubble formation in the microfluidic device, as well as avoiding contact between regions of high electric field and cells.

In addition to improving cellular viability, utilizing small post structures allows better control of cell selectivity by preventing pearl chaining and cell-cell interactions. Cells with different bioelectrical phenotypes are trapped in the main channel at different applied electric field frequencies. By modulating the applied frequency, the device can selectively trap some cells while allowing others to pass through the device. This selectivity allows separation of highly similar cell types in a label-free manner while maintaining high cellular viability such that they can be cultured or further characterized downstream. This method provides more selective and higher viability separation of cells, which allows more closely related and physically similar cells to be separated, while allowing less similar cells to be separated at a much higher efficiency.

Batch separation can be performed by trapping some of the cells while allowing other cells to flow through and be collected in an output tube. After turning off the voltage, trapped cells can be released from their posts and can be collected in another output tube. Accordingly, dielectrophoresis can be used for the size based separation described herein, either to separate large cells, small cells, or activated from inactivated cells.

### Acoustophoretic Isolation

Methods comprising acoustophoresis for separating different cell types can be useful for obtaining one or more target cells from a blood related sample. Acoustophoresis is a phenomenon in which cells, exposed to an acoustic pressure field, are separated based on the characteristics of the cells. See U.S. Pat. No. 10,640,760; See also Dutra, Brian et al. “A Novel Macroscale Acoustic Device for Blood Filtration.” Journal of medical devices vol. 12,1 (2018): 0110081-110087. doi:10.1115/1.4038498. The underlying principle of the acoustic separation is based on the nonuniform acoustic pressure field in the fluid established by an acoustic standing wave. The introduction of a particle in this acoustic pressure field leads to a scattering of the acoustic pressure. The acoustic pressure acting on the surface of the particle then consists of the sum of the incident acoustic standing wave and the scattered wave. The net time averaged force on the particle is determined by integrating the acoustic pressure on the surface of the particle (i.e. acoustic radiation force). In addition to the axial acoustic radiation force component, a three-dimensional acoustic wave also exerts lateral forces on the suspended particle, orthogonal to the axis. An axial component of the acoustic radiation force component directs particles to collect in planes at the pressure nodes or antinodes every half wavelength, determined by a positive or negative acoustic contrast factor, respectively. A lateral component of the acoustic radiation force component collects the cells within the planes to local clusters, where the cells grow in collective size until they reach critical mass and the gravity/buoyancy force causes the cells to sink or rise out of suspension, thus separating the cells.

Separation of cells in a sample can be performed by positive or negative selection of cell types using acoustophoresis and be collected in an output tube. Accordingly, acoustophoresis can be used for the size based separation described herein, either to separate large cells, small cells, or activated from inactivated cells.

The following illustrative examples are representative of embodiments of compositions and methods described herein and are not meant to be limiting in any way.

### Example 1—Determination of Cell Diameters by Axial Light Loss (ALL)

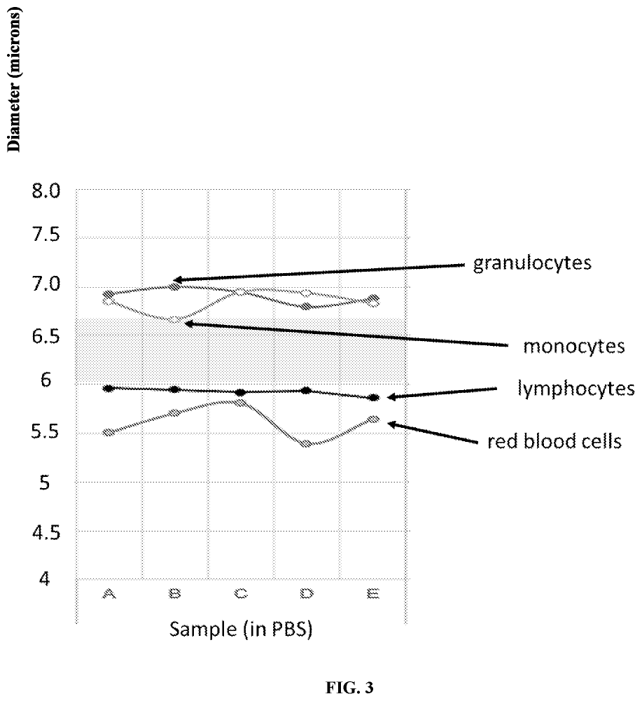

Shown in FIG. 3 is data in which the cell diameters of different cell types from the blood were determined by axial light loss. Different patient samples A, B, C, D, and E in phosphate buffered saline are shown. This data shows that the normal average diameter of a lymphocyte is ˜5.8-6.0 um, which is about 40% lower than current estimates based on microscopy and in the scientific literature. This data fits well with a lower size discriminator set to recover >95% of WBC being about 4 um, and a upper size discriminator of about 6.5 micrometers.

### Example 2—Separation of Lymphocytes from Leukopaks

### Cell Activation and Culture

At day 0, DCS 120×10\hat{ }6 cells from the 4.2+ (e.g., critical size greater than 4.2 micrometers) or from the 4.2+, 6.5− (e.g., critical size greater than 4.2 micrometers and less than 6.5 micrometers performed in series) DLD obstacle array were centrifuged for 5 min at 400×g and the pelleted cells were resuspended in 40 ml of TexMACS media supplemented with 5% Human Serum and Pen/Strep. Control cells from day 0, 4.2+ and 6.5−, fractions were activated with Transact beads (Miltenyi cat #130-111-160) at a 1:100 dilution per manufacturer's instructions. Cells were plated in a 6-well G-Rex plate and cultured at 37° C. and 5% CO₂ in a humidified incubator. See DLD parameters in Table 2 below.

TABLE 2
Array Parameters

| | | Number | | | | | Min Bypass |
|---|---|---|---|---|---|---|---|
| | | of rows | Gap height | Gap width | Post width | Post height | width |
| Description | 1/tilt | in unit | (microns) | (microns) | (microns) | (microns) | on mask |
| D_c 4.3 | 34.4262295 | 60 | 16.5 | 21.8 | 39.2 | 18.5 | 23 |
| D_c 5.5 | 20.6557377 | 36 | 16.5 | 21.8 | 39.2 | 18.5 | 23.98 |
| D_c 6.5 | 19.3846154 | 36 | 16.5 | 25.8 | 39.2 | 18.5 | 28.38 |
| D_c 7.5 | 18.2608696 | 36 | 16.5 | 29.8 | 39.2 | 18.5 | 32.78 |
| D_c 8.5 | 17.260274 | 36 | 16.5 | 33.8 | 39.2 | 18.5 | 37.18 |

| | | | | | Redundancy |
|---|---|---|---|---|---|
| | Length | Width | | | (bumping to edge of |
| Description | (mm) | (mm) | Repeats | Dc | collection channel) |
| D_c 4.3 | 41 | 1.75 | 19.524 | 4.28 | 2.05 |
| D_c 5.5 | 30.9 | 1.9 | 24.524 | 5.46 | 2.37 |
| D_c 6.5 | 30.9 | 1.9 | 24.524 | 6.47 | 2.53 |
| D_c 7.5 | 30.9 | 2.15 | 24.524 | 7.47 | 2.37 |
| D_c 8.5 | 30.9 | 2.15 | 24.524 | 8.47 | 2.51 |

Dc = critical diameter

At day 1, the controls from the 4.2+ and 6.5− were added with 5 ng/ml of each IL7 and IL-15. 20 ml of media were removed from two wells (4.2+6.5−) before cell resuspension and passed thru the 6.5+ DLD obstacle array or 5.5+ DLD obstacle array. Cells from this second processing step were collected (product and waste) and plated in a 6-well G-Rex plate in the presence of IL-7 and 11-15 at 5 ng/ml of each. The cells were cultured at 37° C. and 500 CO₂ in a humidified incubator.

At day 2, two more wells from day 0 (4.2+6.5−) were reduced in volume (−20 mL) and processed further in DLD obstacle arrays with critical sizes of 6.5+ and 5.5+. After processing, the cells were cultured at 37° C./5% CO2 in the presence of IL-7/IL-15 (ng/ml each) in a G-Rex 6 well plate. At day 3 and day 6, cells from all wells were analyzed by flow cytometry.

### Results

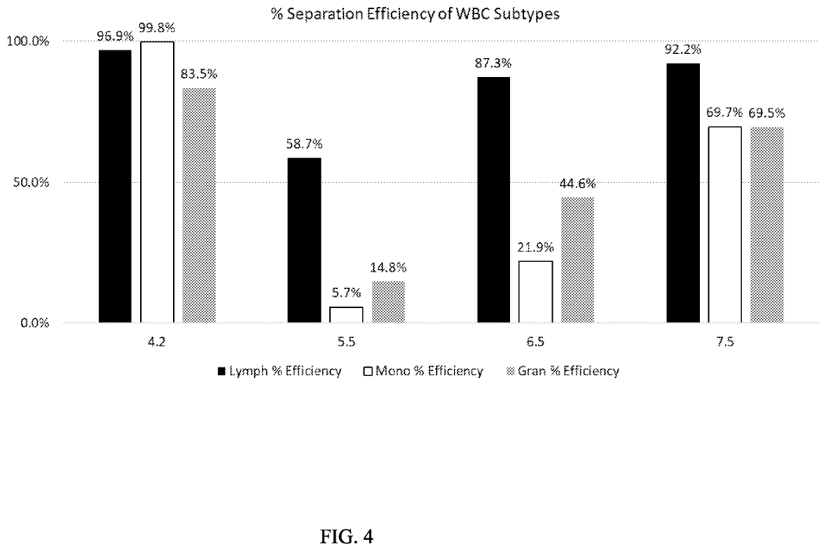

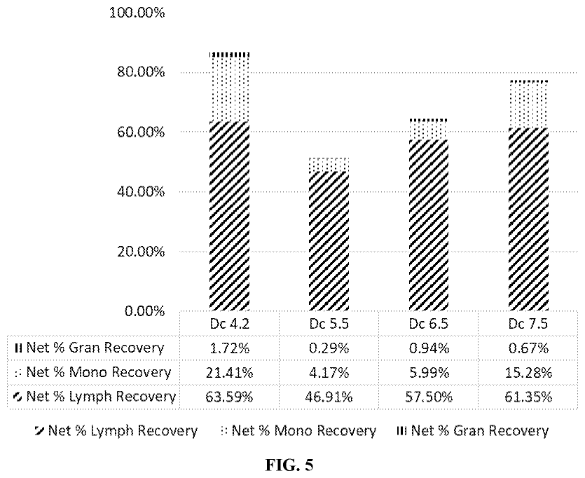

As shown in FIG. 4 different cut-offs of 4.2+, 5.5−, 6.5−, and 7.5− resulted in different separation efficiency. Note: a plus (+) following a number means cells above the stated size were collected and analyzed; whereas a minus (−) following a number means cells below the stated size were collected and analyzed. Collecting cells above 4.2 micrometers resulted in high recovery of lymphocytes, monocytes, and granulocytes, while collecting cells below 5.5 and 6.5 micrometers resulted in high-lymphocyte recovery, while removing a greater proportion of granulocyte and monocyte cells. Table 3 below shows the percentage recovery of each subset as a percentage of total white blood cells recovered and FIG. 5 shows net recovery of different cell subtypes for lymphocytes (diagonal stripes); monocytes (dots); and granulocytes (vertical stripes) subjected to different sizer cut-offs.

TABLE 3

| | % Lymph of | % Mono of | % Gran |
|---|---|---|---|
| Fraction | WBCs | WBCs | of WBCs |
| Dc 4.2+ | 73.2% | 24.1% | 1.6% |
| Dc 5.5− | 94.6% | 4.7% | 0.3% |
| Dc 6.5− | 88.3% | 9.4% | 0.5% |
| Dc 7.5− | 79.6% | 18.0% | 0.8% |

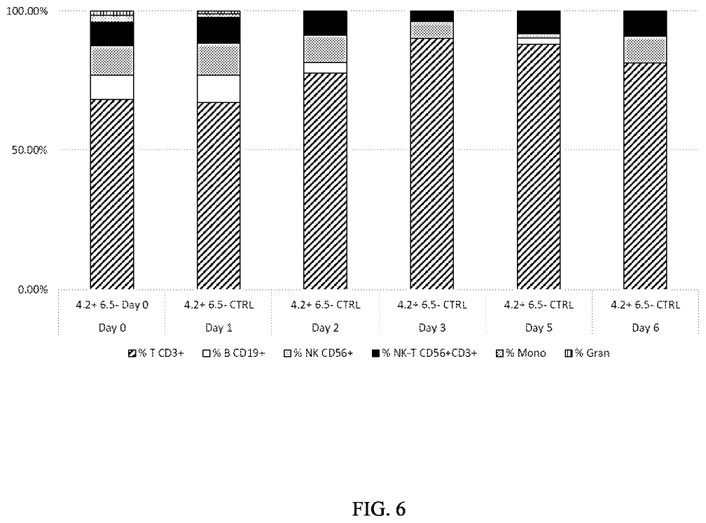

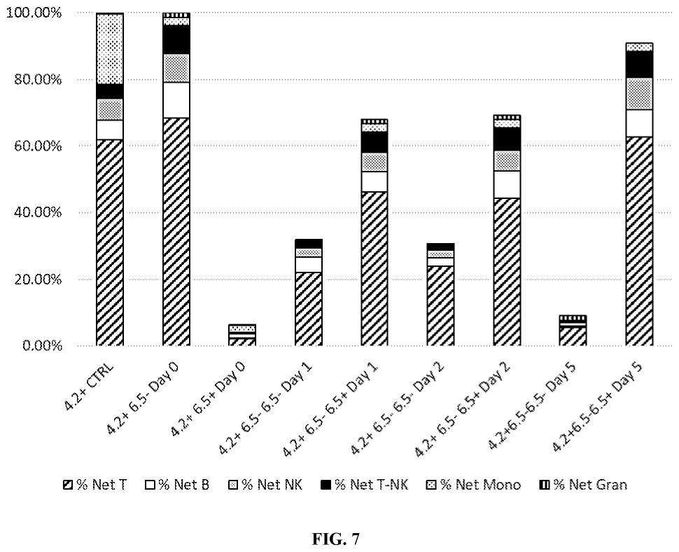

Table 4, FIG. 6 and FIG. 7 shows the lymphocyte subpopulations isolated from a 4.2+ enrichment followed by activation (FIG. 7), or not (FIG. 6), for the specified amount of time followed by 6.5− enrichment.

TABLE 4

| Day of | | | | | | | |
|---|---|---|---|---|---|---|---|
| Run | Sample Type | % T | % B | % NK | % NK-T | % Mono | % Gran |
| 0 | 4.2+ 6.5− D0 | 68.34% | 8.59% | 10.68% | 8.38% | 2.55% | 1.45% |
| 1 | 4.2+ 6.5− CTRL | 67.19% | 9.79% | 11.50% | 9.28% | 1.19% | 1.05% |
| 2 | 4.2+ 6.5− CTRL | 77.74% | 3.74% | 9.86% | 7.99% | 0.22% | 0.45% |
| 3 | 4.2+ 6.5− CTRL | 90.10% | 0.08% | 6.07% | 3.57% | 0.01% | 0.17% |
| 5 | 4.2+ 6.5− CTRL | 88.09% | 2.31% | 1.43% | 8.14% | 0.03% | 0.00% |
| 6 | 4.2+ 6.5− CTRL | 81.35% | 0.04% | 9.46% | 9.07% | 0.03% | 0.05% |

Combining a lower and an upper size cut-off, for example, a 4.0+, 4.1+, or 4.2+ cutoff combined with a 6.5− cut-off results in a cell population highly enriched for lymphocytes with minimal amounts of red blood cells or platelets. For example, as shown in FIG. 6 just after isolation (day 0) the enriched cell population was greater than 95% lymphocytes and less than 5% (granulocytes and monocytes), by day 3 after culture granulocytes and monocytes make up less than 0.5% of the total cells. This significantly exceeds lymphocyte purity seem with the current standard of elutriation. See e.g., Stroncek et al. “Elutriated lymphocytes for manufacturing chimeric antigen receptor T cells. “J Trans Med (2017)15:59. In addition, enrichment described in this example has high-throughput, is scalable for manufacturing, and avoids contacting therapeutic cells with toxic density gradient media.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A method of enriching lymphocytes from a biological sample, the method comprising:
a. separating large cells and small cells from the biological sample by a size-based selection method to obtain lymphocytes, wherein the large cells comprise granulocytes monocytes, or a combination thereof, and the small cells comprise platelets, red blood cells, or a combination thereof;
b. contacting the lymphocytes with a size increasing agent to obtain size increased lymphocytes; and optionally
c. separating non-size increased lymphocytes from the size increased lymphocytes to obtain size increased enriched lymphocytes, thereby obtaining enriched size increased lymphocytes.

2. The method of claim 1, further comprising separating platelet derived microvesicles, microparticles, or exosomes.

3-6. (canceled)

7. The method of claim 1, wherein the large cells possess a diameter of 6.5 micrometers or greater.

8. (canceled)

9. The method of claim 1, wherein the large cells comprise monocytes, granulocytes, dendritic cells, or a combination thereof.

10-11. (canceled)

12. The method of claim 1, wherein the small cells possess a diameter of 5 micrometers or less.

13. The method of claim 1, wherein the small cells comprise platelets and red blood cells.

14. The method of claim 1, wherein the size increasing agent comprises an activating agent.

15. The method of claim 1, wherein the lymphocytes comprise T cells, B cells, NK cells, or a combination thereof.

16. (canceled)

17. The method of claim 1, wherein the size increasing agent comprises an agent selected from the list consisting of a CD3 binding antibody, a CD28 binding antibody, a CD49d binding antibody, Concanavalin-A, and combinations thereof.

18. (canceled)

19. The method of claim 1, wherein the size increasing agent is selected from the list consisting of a IgM binding antibody, a IgD binding antibody, a CD 154 binding antibody, a CpG oligonucleotide, LPS, single stranded RNA, imiquimod, and combinations thereof.

20. (canceled)

21. The method of claim 1, wherein the size increasing agent is selected from the list consisting of a CD335(NKp46) binding antibody, a CD2 binding antibody, LPS, peptidoglycan, a mIR-150 microRNA, and combinations thereof.

22. (canceled)

23. The method of claim 1, wherein the non-size increased lymphocytes possess a diameter of 8.0 micrometers or less.

24. The method of claim 1, wherein the size-based selection method comprises deterministic lateral displacement.

25. The method of claim 1, wherein the size based selection method comprises acousticpheresis.

26. The method of claim 1, wherein the large cells are removed before the small cells are removed.

27. The method of claim 1, wherein the small cells are removed before the large cells are removed.

28. The method of claim 1, wherein the small cells and the large cells are removed simultaneously.

29. The method of claim 1, wherein separating non-size increased lymphocytes from the size increased lymphocytes is performed using a size-based selection method.

30. The method of claim 1, wherein separating non-size increased lymphocytes from the size increased lymphocytes occurs at least 4 hours and prior to 60 hours after contacting the lymphocytes with the size increasing agent.

31. (canceled)

32. The method of claim 1, wherein separating the small cells from the biological sample does not use a reagent with affinity for the small cells.

33. (canceled)

34. The method of claim 1, wherein separating the large cells from the biological sample does not use a reagent with affinity for the large cells.

35. (canceled)

36. The method of claim 1, wherein separating the non-size increased lymphocytes from the size increased lymphocytes does not use a reagent with affinity for the inactivated lymphocytes.

37. (canceled)

38. The method of claim 1, wherein the non-size increased lymphocytes comprise B cells, T cells, NK cells, or NKT cells.

39-41. (canceled)

42. The method of claim 1, wherein the enriched size increased lymphocytes comprise B cells, T cells, NK cells, NKT cells, or combinations thereof.

43-45. (canceled)

46. The method of claim 1, further comprising genetically engineering the enriched size increased lymphocytes or the size increased lymphocytes to produce genetically engineered size increased lymphocytes.

47. The method of claim 46, wherein the genetically engineered size increased lymphocytes express a chimeric antigen receptor.

48. The method of claim 46, wherein the genetically engineered size increased lymphocytes are genetically engineered by use of a virus, plasmid DNA, or mRNA.

49. The method of claim 1, wherein the size-based separation comprises a microfluidic device configured for deterministic lateral displacement.

50. The method of claim 49, wherein the microfluidic device comprises a plurality of arrays comprising a plurality of obstacles arranged into rows running approximately perpendicular to a direction of fluid flow and columns running approximately parallel to the direction of fluid flow, wherein the columns are offset from the direction of fluid flow by a tilt angle.

51. The method of claim 50, wherein the device comprises at least 50 arrays of obstacles.

52-55. (canceled)

56. The method of claim 50, wherein each obstacle of the plurality of obstacles has a diamond, circular, ellipsoid, or hexagonal shape.

57. The method of claim 50, wherein each obstacle of the plurality of obstacles has a P1 length approximately parallel to the direction of fluid flow that is longer than a P2 length approximately perpendicular to the direction of fluid flow.

58. (canceled)

59. The method of claim 57, wherein P1 is about 10 μm to about 60 μm and P2 is about 10 μm to about 30 μm.

60. (canceled)

61. The method of claim 57, wherein P1 is 50% to 150% longer than P2.

62. The method of claim 50, wherein the obstacles in a column of obstacles are separated by a G1 gap of about 20 to 35 μm and the obstacles in a row of obstacles are separated by a G2 gap of about 15 to 20 μm.

63-65. (canceled)

66. The method of claim 50, wherein the microfluidic device comprises
a. a first plurality of arrays comprising a plurality of obstacles arranged into rows running approximately perpendicular to a direction of fluid flow and columns running approximately parallel to the direction of fluid flow, wherein the columns are offset from the direction of fluid flow by a tilt angle; and
b. a second plurality of arrays comprising a plurality of obstacles arranged into rows running approximately perpendicular to a direction of fluid flow and columns running approximately parallel to the direction of fluid flow, wherein the columns are offset from the direction of fluid flow by a tilt angle.

67-71. (canceled)

72. The method of claim 50, wherein a buffer flows continuously through the microfluidic device.

73. The method of claim 50, wherein the flow rate through the microfluidic device is at least about 500, 600, 700, 800, 900, or 1,000 mL per hour.

74. The method of claim 50, wherein the microfluidic device operates in oscillatory flow conditions.
